SEQUENCE LISTING (11 sequences)

SEQ ID NO: 1 - DNA, 1191 bp, Homo sapiens
atggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctag

SEQ ID NO: 2 - DNA, 20 bp, Artificial Sequence
  Primer
gccatcaacgaccccttcat

SEQ ID NO: 3 - DNA, 20 bp, Artificial Sequence
  Primer
agatggtgatgggtttcccg

SEQ ID NO: 4 - DNA, 20 bp, Artificial Sequence
  Primer
ccttaagggccagctacacc

SEQ ID NO: 5 - DNA, 20 bp, Artificial Sequence
  Primer
agcgttggtgttgtacgtct

SEQ ID NO: 6 - DNA, 20 bp, Artificial Sequence
  Primer
tggtgagacgtggaaacctg

SEQ ID NO: 7 - DNA, 20 bp, Artificial Sequence
  Primer
cttgggtccctcgactccta

SEQ ID NO: 8 - DNA, 20 bp, Artificial Sequence
  Primer
ggtggtgaatagactccggc

SEQ ID NO: 9 - DNA, 20 bp, Artificial Sequence
  Primer
agctcgtcacaattggggtt

SEQ ID NO: 10 - DNA, 20 bp, Artificial Sequence
  Primer
agctcgtcacaattggggtt

SEQ ID NO: 11 - DNA, 20 bp, Artificial Sequence
  Primer
gctccggcctacaaatctca

(19) United States
(12) Patent Application Publication
(10) Pub. No.: US 2021/0052776 A1
(43) Pub. Date: Feb. 25, 2021

(54) MESENCHYMAL STEM CELLS OR STROMAL CELLS HARBORING MODIFIED RNAS ENCODING VEGF AND BMP POLYPEPTIDES

(71) Applicants: Nevin Witman, Reading, PA (US); Kenneth R. Chien, Cambridge, MA (US); Wei Fu, Shanghai (CN)

(72) Inventors: Nevin Witman, Reading, PA (US); Kenneth R. Chien, Cambridge, MA (US); Wei Fu, Shanghai (CN)

(21) Appl. No.: 16/918,979

(22) Filed: Jul. 1, 2020

Related U.S. Application Data
(60) Provisional application No. 62/869,941, filed on Jul. 2, 2019.

(51) Int. Cl. A61L 27/38; A61K 35/28; A61K 35/51; A61K 35/50; A61K 9/00; A61P 19/00; C07K 14/51; C07K 14/475; C12N 5/0775; A61L 27/54

(52) U.S. Cl. CPC A61L 27/3821; A61K 35/28; A61K 35/51; A61K 35/50; A61K 9/0019; A61P 19/00; A61L 2300/414; C07K 14/475; C12N 5/0663; A61L 27/3847; A61L 27/54; A61L 2430/02; C07K 14/51

(57) ABSTRACT

This disclosure relates to compositions including mesenchymal stem or stromal cells (MSCs) that harbor one or more modified RNA molecules encoding a bone morphogenetic protein (BMP), e.g., human BMP, and one or more modified RNA molecules encoding vascular endothelial growth factor (VEGF), e.g., human VEGF or VEGF-A, or first and second separate pluralities of MSCs, wherein each MSC in the first plurality of MSCs harbors one or more modified RNA molecules encoding BMP, and wherein each MSC in the second plurality of MSCs harbors one or more modified RNA molecules encoding VEGF; and a carrier, e.g., a solid or semi-solid carrier. The disclosure also relates to methods and uses of these compositions to treat bone defects.

### CROSS-REFERENCE TO RELATED APPLICATIONS

This application claims priority to U.S. Provisional Patent Application Ser. No. 62/869,941, filed on Jul. 2, 2019, the entire contents of which are hereby incorporated by reference.

### FIELD OF THE INVENTION

This disclosure relates to modified RNA molecules encoding Vascular Endothelial Growth Factor (VEGF) polypeptides and Bone Morphogenetic Protein (BMP) polypeptides, and to mesenchymal stem or stromal cells (MSCs) harboring modified RNA molecules encoding these polypeptides for use in therapies for bone defects.

### BACKGROUND OF THE INVENTION

Non-healing bone defects can develop following severe trauma, nonunion fractures, tumor resection, or craniomaxillofacial surgery. Several therapeutic approaches have been developed to treat non-healing bone defects including bone grafts (e.g., autograft or allograft), mesenchymal stem cells (MSCs), tissue-engineered bone grafts (TEBG) (Zhang et al., Biomaterials, 31:608-620 (2010); Wang et al., Tissue Eng., Pt A 21:2346-2355 (2015)), composites including biomaterials and growth factors (e.g., human bone morphogenetic protein 2 (BMP-2), a growth differentiation factor) (Chen et al., J. Biomed. Mater. Res., A 106:706-717 (2018); Wang et al., Int. J. Nanomed., 12:7721-7735 (2017)), and gene therapy (Im, G. I. Journal of Biomedical Materials Research, Part A 101:3009-3018 (2013)).

Therapeutic approaches involving recombinant growth factors, e.g., human vascular endothelial growth factor (VEGF-A) and BMP-2, have high manufacturing costs and must be administered at supraphysiological dosages to achieve successful therapeutic efficacy due to the short half-lives of the proteins (Carmeliet, Curr. Interv. Cardiol., Rep 1:322-335 (1999)) and to the low bioavailability in vivo (Tannoury et al., Spine J., 14:552-559 (2014)). Patients can suffer adverse effects due to the administration of high dose of recombinant growth factors. For example, patients receiving high dose recombinant BMP-2 preparation in the AMPLIFY® product exhibited a higher rate of new malignancy occurrence compared to patients in the control group (Carragee et al., Spine J., 11:471-491 (2011); Carragee et al., Cancer Risk After J. Bone Joint Surg. Am., 95a:1537-1545 (2013)).

Gene therapy can also be used to deliver recombinant proteins. For example, transgenes can be locally delivered to select tissues. Additionally, transgenes can be targeted to specific cell-types or tissues and further expressed in subsets of those cells when operably linked to promoters that drive expression in specific cell-types (Evans, C. H., Adv. Drug Deliv. Rev., 64:1331-1340 (2012)). Consequently, gene therapy represents a potential therapeutic option for bone healing (Wegman et al., Biotechnol. Genet. Eng., 29:206-220 (2013)). However, gene-based therapies can cause adverse effects to patients—e.g., induction of DNA mutations and induction of inflammatory responses—e.g., activation of an immune response (Muruve et al., Hum. Gen. Ther., “the innate immune response to adenovirus vectors” (2004)). In addition, it is difficult to regulate dosage with gene therapy, and the effect can be far longer lasting than desired (Bouard et al., Br. J. Pharmacol., “Viral vectors: from virology to transgene expression” (2009)).

For example, a multicenter randomized clinical study revealed high doses of BMP2 used in posterolateral spinal bone fusions gave rise to higher incidences of carcinoma in patients treated with BMP2 recombinant proteins than those with autograft controls (Carragee et al., “Cancer risk after use of recombinant bone morphogenetic protein-2 for spinal arthrodesis,” J. Bone Joint Surg. Am., 95:1537 (2013)). Furthermore, biological research has found links between BMP and carcinoma, including BMP ligand up-regulation in breast and pancreatic carcinoma (Ye et al., “Bone morphogenetic proteins in development and progression of breast cancer and therapeutic potential,” Int. J. Mol. Med., 24(5):591-7 (2009); Kleeff et al., “Bone morphogenetic protein 2 exerts diverse effects on cell growth in vitro and is expressed in human pancreatic cancer in vivo,” Gastroenterology, 116(5):1202-16 (1999)), respectively, as well as the role for BMP2 to increase the proliferation of multiple cancer cell types including lung (Langenfeld et al., “Bone morphogenetic protein 2 stimulation of tumor growth involves the activation of Smad-1/5,” Oncogene, 25:685-692 (2006)), breast (Jin et al., “BMP2 promotes migration and invasion of breast cancer cells via cytoskeletal reorganization and adhesion decrease: an AFM investigation,” Appl. Microb. Biotechnol., 93(4):1715-23 (2012)), and pancreatic (Kleeff et al., 1999) cancers.

Another major inherent risk in association with BMP2 is the induction of inflammation. A recent study elegantly demonstrated high doses of BMP2 administration were effectively able to fuse bone defects, however such a dose was met with cyst-like bone and soft tissue swelling (Zara et al., “High Doses of Bone Morphogenetic protein 2 induce structurally abnormal bone and inflammation in vivo,” Tissue Eng. Part A, 17(9-10):1389-99 (2011)). When lower doses were applied to the femoral segmental defects in rats, the BMP2 was unable to induce full fusions, but rendered no adverse effects.

Given the challenges associated with known bone regeneration therapeutic strategies, there is an unmet need for new therapeutic strategies that confer fewer adverse effects in patients and that are cost-efficient.

### SUMMARY OF THE INVENTION

The present disclosure is based, at least in part, on the discovery that if one uses mesenchymal stem/stromal cells (MSCs), e.g., bone marrow-derived MSCs (BMSCs), adipose/fat-derived MSCs, peripheral blood-derived MSCs, umbilical cord-derived MSCs, or placental-derived MSCs, e.g., which can be either autologous or allogeneic cells, wherein the MSCs harbor modified RNA molecules that encode VEGF (e.g., VEGF-A, B, C) polypeptides and BMP (e.g., BMP-2/3/4/5/6/7/8/9) polypeptides, one can deliver a controlled dosage of these proteins for a controlled length of time to provide an improved therapy to heal bone defects, e.g., fractures in normal bone and non-healing bone defects including, but not limited to, non-union fractures, osteoporosis related fracture prevention and repair; and osteonecrosis prevention and repair (avascular necrosis).

In one aspect, the disclosure provides compositions including or consisting of or consisting essentially of (a) a mesenchymal stem or stromal cell (MSC) composition comprising either: (i) a plurality of MSCs, wherein each MSC harbors one or more modified RNA molecules encoding bone morphogenetic protein (BMP), e.g., human BMP, e.g., BMP-1, BMP-2, BMP-3, BMP-4, BMP-5, BMP-6, BMP-7, BMP-8a, and BMP-8b, and BMP-9, and their respective isoforms, and one or more modified RNA molecules encoding vascular endothelial growth factor (VEGF), e.g., human VEGF, e.g., VEGF-A, -B, -C, -D, and -E, and their respective isoforms, e.g., VEGF-A isoforms (e.g., 165, 121, 189, or 206), or human placental growth factor (PlGF) and its isoforms, which are members of the VEGF superfamily, or (ii) first and second separate pluralities of MSCs, wherein each MSC in the first plurality of MSCs harbors one or more modified RNA molecules encoding BMP, e.g., human BMP or variations as noted in (i), and wherein each MSC in the second plurality of MSCs harbors one or more modified RNA molecules encoding VEGF, e.g., human VEGF, e.g., VEGF-A or variations as noted in (i); and (b) a carrier, e.g., a solid or semi-solid carrier.

In various implementations, the carrier includes or consists of a gel, hydrogel, paste, or a solid carrier, e.g., a physical carrier in a gel, hydrogel, or paste formulation. For example, the carrier can be or include or consist of one or more bioceramics, bioactive glasses, polymer components/nanofibers (e.g., polyfumarates, polycaprolactones, polylactic acid (PLA), polyglycolic acid (PGA), copolymers of PLA and PGA (PLGA)), nanomaterials (e.g., nanotubes), hydroxyapatite (HA) (e.g., in the form of crystals or other), collagen fibers/sponges, collagen molecules, non-toxic lipid nanoparticles, and/or gel/silica-based hydrogels.

In some implementations, the VEGF polypeptide is a VEGF-A polypeptide and the BMP polypeptide is a BMP-2 polypeptide.

In different implementations, the MSCs are bone marrow-derived MSCs (BMSCs), adipose/fat-derived MSCs, peripheral blood-derived MSCs, umbilical cord-derived MSCs, or placental-derived MSCs. In some implementations, the MSCs are allogenic cells or autologous cells.

In certain embodiments, the MSCs harboring the modified RNA molecules are prepared by transient transfection or electroporation with the modified RNA molecules, as described herein. For example, the MSCs harboring modified RNA molecules can be prepared by exposure to lipid nanoparticles including the modified RNA molecules.

In some implementations, the concentration of the modRNAs used in the cells is in the range of 1 pg to 10 pg per cell. For example, a total dose may be 10 mg for, e.g., 1 billion cells.

In some implementations, the total number of cells in the composition is in the range of about 20×10⁴ to about 2 to 120×10⁶. In certain embodiments, the dose of mRNA transcripts within the MSCs is about 1 to 10 pg/cell to 1 ng/cell.

In certain implementations, the composition of cells includes about 25 μg of modRNA in about 250,000 cells at a dose of 10 pg/cell, and secretes about 10 to 100 ng of the BMP and VEGF polypeptides over a period of 5 days, per composition. The amount of protein secreted is largely dependent on two factors, amount of mRNA loaded into each cell, and the number of cells in the composition.

In some embodiments, the composition includes about 25 μg to about 2.5 mg of modRNA in about 2.5 to 25×10⁶ cells at a dose of 1-100 pg/cell, and secretes about 1 ng to 10 mg of the BMP and VEGF polypeptides per composition (levels of secretion is dependent on number of cells in the composition), e.g., over a period of 5 days. For example, 2 million cells may produce 1 mg of protein and 25 million cells would produce about 10 mg of protein, or more.

In different implementations, a ratio of BMP to VEGF is in a range of 10:1 to 1:1, e.g., 9:1, 8:1, 7:1, 6: 1, 5:1, 4:1, 3:1, 2:1, or 1:1. For example, the ratio of BMP-VEGF can be about 3:1, 4:1, 5:1, or 6:1.

In another aspect, the disclosure provides any of the compositions described herein for use in methods of treating a bone defect, as well as methods of treating a bone defect, e.g., a non-healing bone defect, in a subject, wherein the methods include, optionally, identifying a subject with a bone defect; and administering to the bone defect any of the compositions described herein. In these methods, the bone defect can be a site of osteoporosis, a bone tumor, a bone break, a site of bone trauma, a nonunion bone fracture, a bone tumor resection, or a bone affected by craniomaxillofacial surgery.

In some implementations of these methods, the carrier can be or include a gel, hydrogel, paste, or a solid carrier, e.g., a physical carrier in a gel, hydrogel, or paste formulation. For example, the carrier can be one or more bioceramics, bioactive glasses, polymer components/nanofibers (e.g., polyfumarates, polycaprolactones, polylactic acid (PLA), polyglycolic acid (PGA), copolymers of PLA and PGA (e.g., PLGA)), nanomaterials (e.g., nanotubes), hydroxyapatite (HA) (e.g., in the form of crystals or other), collagen fibers/sponges, collagen molecules, non-toxic lipid nanoparticles, and/or gel/silica-based hydrogels.

In some implementations of these methods, the VEGF polypeptide is a VEGF-A, VEGF-B, VEGF-C, VEGF-D, or VEGF-E, or PlGF polypeptide, and the BMP polypeptide is a BMP-2, BMP-1, BMP-3, BMP-4, BMP-5, BMP-6, BMP-7, BMP-8a/b, BMP-9 polypeptide, and the MSCs are bone marrow-derived MSCs (BMSCs), adipose/fat-derived MSCs, peripheral blood-derived MSCs, umbilical cord-derived MSCs, or placental-derived MSCs. In certain embodiments, the MSCs are allogenic cells or autologous cells.

In certain implementations, the composition is administered by injection to the bone defect. In other implementations, the composition is administered by vascular catheterization. In certain embodiments, the composition is administered using MSC engineered to migrate to a site areas of the bone defect. In yet other embodiments, the composition is administered by a surgical procedure to render access to the bone defect, e.g., bone core decompression.

As used herein, the term “nucleic acid” refers to any composition having a polymer of nucleotides linked via phosphodiester bond.

As used herein, the term “messenger RNA” (mRNA) refers to any polynucleotide that encodes a polypeptide of interest and that is capable of being translated to produce the encoded polypeptide of interest in vitro, in vivo, in situ, or ex vivo.

As used herein, the term “modified RNA” refers to an RNA further including one or more modifications relative to the standard RNA nucleotide chain. Examples of modifications include, but are not limited to, modifications to a sugar, a nucleobase (e.g., replacement or substitution of an atom with a substituted amino, a substituted thiol, a substituted alkyl, or a halogen), or an internucleoside linkage. In some embodiments, the modified RNA avoids the innate immune response upon administration to a subject. In some embodiments, the half-life of the modified RNA within a subject is extended compared to an unmodified RNA.

As used herein, the term “harboring” refers to a cell having a component internally—e.g., in the cytoplasm or in an intracellular compartment. In some embodiments, the component is a modified RNA and the cell is made to harbor the modified RNA by methods known in the art including, but not limited to, electroporation, transfection (e.g., methods using cationic-lipid transfection reagents), and lipid nanoparticle encapsulation, e.g., of modified RNAs.

As used herein, the term “administering” refers to delivering to a subject a composition, e.g., a pharmaceutical composition, or a formulation, e.g., a pharmaceutical formulation, including at least one cell harboring one or more modified RNA encoding BMP-2 and one or more modified RNA encoding VEGF-A into a subject by a method or route that results in at least partial localization of the composition or formulation at a desired site or tissue location within the subject, e.g., at the site or location of a bone defect.

As used herein, the terms “formulation” or “pharmaceutical formulation” refers to a composition including one or more active pharmaceutical ingredients (e.g., a cell harboring one or more modified RNAs) and one or more pharmaceutically acceptable carriers, diluents, and/or excipients.

As used herein, the term “effective amount” refers to the amount of therapeutic agent, pharmaceutical composition, or pharmaceutical formulation, sufficient to reduce at least one or more symptom(s) of the disease or disorder, or to provide the desired effect.

As used herein, the term “bone defect” refers to acute fractures in normal bone, osteoporosis related fracture repair and prevention; osteonecrosis/avascular necrosis related fracture repair and prevention, oral maxillofacial reconstruction and/or spinal fusion.

As used herein, the term “subject” means any animal or human, including mammals, such as humans, primates, dogs, cats, horses, cows, pigs, goats, rabbits, rats, mice, and other domesticated mammals.

As used herein, “autologous cells” are cells that are removed from a subject and can then be engineered, e.g., by inserting modified RNA, and then these cells can either be expanded through in vitro mechanisms and re-introduced back into the same subject, or immediately introduced back to the same subject following modified mRNA insertion.

As used herein, “allogeneic cells” are cells that with respect to a first subject are removed from a second subject, and can then be engineered, e.g., by inserting modified RNA, and then expanded through in vitro mechanisms and then these cells from the second subject are introduced into the first subject.

As used herein, “vascular endothelial growth factor” or “VEGF” means any polypeptide that is an essential growth factor for vascular endothelial cells and includes VEGF family members, for example, VEGF-A, placenta growth factor (PGF), VEGF-B, VEGF-C, VEGF-D, VEGF-E, and VEGF-F, and their respective isoforms. The VEGF polypeptides can be human polypeptides and/or recombinant forms of these polypeptides.

As used herein, “bone morphogenic protein” or “BMP” means any polypeptide that is a growth factor for bone and/or cartilage and includes BMP family members, for example, BMP-1, BMP-2, BMP-3, BMP-4, BMP-5, BMP-6, BMP-7, BMP-8a, BMP-8b, and BMP-9, and their respective isoforms. The BMP polypeptides can be human polypeptides and/or recombinant forms of these polypeptides.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### SEQUENCE LISTING

The instant application contains a Sequence Listing, which has been submitted electronically in ASCII format and is hereby incorporated by reference in its entirety. Said ASCII copy, created on Aug. 27, 2020, is named “Sequence Listing” and is 3.66 KB in size.

### BRIEF DESCRIPTION OF THE DRAWINGS

This patent or application contains at least one drawing executed in color. Copies of this patent or patent application publication with color drawing(s) will be provided by the Office upon request and payment of the necessary fee.

FIG. 1A is a series of related representative fluorescence microscopy images of bone marrow stem cells (BMSCs) co-transfected with modified RNAs encoding green fluorescent protein (GFP) and red fluorescent protein (mCherry) and a merge image depicting the possibility of multiplexing (expressing) 2 or more modified mRNA transcripts in the same cells.

FIG. 1B is a western blot showing VEGF-A protein expression relative to β-actin expression at 24 hours in BMSCs transfected with modified RNA encoding VEGF-A.

FIG. 1C is a bar graph showing quantification of VEGF-A protein expression relative to β-actin expression at 24 hours in BMSCs transfected with modified RNA encoding VEGF-A.

FIG. 1D is a bar graph showing quantification of VEGF-A protein expression relative to β-actin expression over time in BMSCs transfected with modified RNA encoding VEGF-A.

FIG. 1E is a representative western blot showing BMP-2 protein expression relative to β-actin expression at 24 hours in BMSCs transfected with modified RNA encoding BMP-2.

FIG. 1F is a bar graph showing quantification of BMP-2 protein expression relative to β-actin expression at 24 hours in BMSCs transfected with modified RNA encoding BMP-2.

FIG. 1G is a bar graph showing quantification of BMP-2 protein expression relative to (3-actin expression over time in BMSCs transfected with modified RNA encoding BMP-2.

FIG. 2A are a series of representative fluorescence microscopy images of BMSCs taken 24 hours post-transfection with GFP modRNA complexes and stained with DAPI. The applied modRNA dose was 10 pg/cell. The scale bars represent 125 μm.

FIG. 2B is a graph showing GFP expression of BMSCs after transfection with GFP modRNA as determined by flow cytometry.

FIG. 2C is a bar graph of representative flow cytometry histograms for BMSCs 24 hours post-transfection with GFP modRNA.

FIG. 2D is a graph showing the effect of modRNA complexes on BMSC proliferation.

FIGS. 2E and 2F are bar graphs showing qPCR determination of gene expression in BMSCs transfected with VEGF-A and BMP2 modRNA for 24 hours, respectively.

FIGS. 2G and 2H are graphs of the time course of BMP2 and VEGF-A protein expression and accumulation from BMSCs transfected with modBMP2 and modVEGF-A complexes, respectively.

FIG. 3A is a bar graph showing quantification of alkaline phosphatase activity using a colorimetric assay at 7 days post-transfection; (black) transfected with no RNA; (blue) transfected with modified RNA encoding VEGF-A; (green) transfected with modified RNA encoding BMP-2; (red) co-transfected with modified RNA encoding VEGF-A and modified RNA encoding BMP-2.

FIG. 3B is a bar graph showing quantification of calcium deposition using alizarin red staining at 14-days post-transfection; (black) transfected with no RNA; (blue) transfected with modified RNA encoding VEGF-A; (green) transfected with modified RNA encoding BMP-2; (red) co-transfected with modified RNA encoding VEGF-A and modified RNA encoding BMP-2.

FIGS. 3C-3F are bar graphs showing quantification of (C) alkaline phosphatase (ALP), (D) Collagen Type I (COL1), (E) Osteocalcin (OCN), (F) Runt-related transcription factor 2 (RunX2) at 7 days post-transfection determined by qRT-PCR analysis; (black) transfected with no RNA; (blue) transfected with modified RNA encoding VEGF-A; (green) transfected with modified RNA encoding BMP-2; (red) co-transfected with modified RNA encoding VEGF-A and modified RNA encoding BMP-2.

FIGS. 3G-3J are bar graphs showing quantification of (G) ALP, (H) COL1, (I) OCN, (J) (RunX2) at 7 days post-transfection determined by western blotting analysis; (black) transfected with no RNA; (blue) transfected with modified RNA encoding VEGF-A; (green) transfected with modified RNA encoding BMP-2; (red) co-transfected with modified RNA encoding VEGF-A and modified RNA encoding BMP-2.

FIG. 4A is a series of two bar graphs showing the quantitative analysis of the tube formation assay between VEGF-A modRNA transfected and untransfected groups, P<0.05.

FIG. 4B are a series of four daily IVIS images and recordings demonstrating positive luciferase expression following modRNA-treated BMSC transplantation.

FIG. 4C is a bar graph showing positive luciferase expression following modRNA-treated BMSC transplantation. Luciferase levels were measured as photons/sec/cm2/sr. Positive luciferase protein expression could be assayed for three days. At day 4, protein expression dropped to baseline values.

FIG. 5A is a schematic of the experimental design for BMSCs harboring modified RNA encoding VEGF-A and modified RNA encoding BMP-2 combined with biomaterials to enhance bone healing in a rat cranial defect model.

FIG. 5B is a representation of the cranial defect surgery process including stages 1 to 5.

FIG. 5C are a series of five pairs of representative images reconstructed from 3D μCT scans showing bone tissue regeneration at 4 weeks and 12 weeks after treatment for Col, Untransfected, VEGF-A transfected, hBMP-2 transfected, and hBMP-2 and VEGF-A transfected mice.

FIGS. 5D-5E are bar graphs showing quantified bone volume fraction (BV/TV) and bone volume (BV) of regenerated bone at 4 weeks post-implantation of BMSCs harboring modified RNA encoding VEGF-A and modified RNA encoding BMP-2.

FIGS. 5F-5G are bar graphs showing quantified bone volume fraction (BV/TV) and bone volume (BV) of regenerated bone at 12 weeks post-implantation of BMSCs harboring modified RNA encoding VEGF-A and modified RNA encoding BMP-2.

FIGS. 6A-6E are representative microscopic images of hematoxylin and eosin (H&E) stained tissues of the cranial defect location at 4-weeks weeks post-implantation of BMSCs harboring select modified RNA molecules. The arrows indicate the complete bridging of new bone (yellow arrow), callus tissue (blue arrow), and fibrous connective tissue.

FIGS. 6F-6J are representative microscopic images of hematoxylin and eosin (H&E) stained tissues of the cranial defect location at 12-weeks weeks post-implantation of BMSCs select modified RNA molecules. The arrows indicate the complete bridging of new bone (yellow arrow), callus tissue (blue arrow), and fibrous connective tissue.

FIGS. 6K-60 are representative microscopic images of masons trichrome stained tissues of the cranial defect location at 4-weeks post-implantation of BMSCs harboring select modified RNA molecules. The arrows indicate the complete bridging of new bone (yellow arrow) callus tissue; (red arrow) fibrous connective tissue.

FIGS. 6P-6T are representative microscopic images of Masson's trichrome stained tissues of the cranial defect location at 12-weeks post-implantation of BMSCs harboring select modified RNA molecules. The arrows indicate the complete bridging of new bone (yellow arrow) callus tissue; (red arrow) fibrous connective tissue.

FIGS. 7A-7B are bar graphs showing quantified osteocalcin (OCN) positive cells at the site of regenerating bone at 4 and 12 weeks post-implantation of BMSCs harboring select modified RNA molecules; (black) transfected with no RNA; (blue) transfected with modified RNA encoding VEGF-A; (green) transfected with modified RNA encoding BMP-2; (red) co-transfected with modified RNA encoding VEGF-A and modified RNA encoding BMP-2.

FIGS. 7C-7D are bar graphs showing quantified CD-31 immunolabeling at the site of regenerating bone at 4 and 12 weeks post-implantation of BMSCs harboring select modified RNA molecules; (black) transfected with no RNA; (blue) transfected with modified RNA encoding VEGF-A; (green) transfected with modified RNA encoding BMP-2; (red) co-transfected with modified RNA encoding VEGF-A and modified RNA encoding BMP-2.

FIGS. 8A-8D are bar graphs showing quantification of (A) ALP, (B) COL1, (C) OCN, (D) RunX2 expression measured by qRTPCR in the regenerating bone at 4 weeks post-implantation of BMSCs harboring select modified RNA molecules; (black) transfected with no RNA; (blue) transfected with modified RNA encoding VEGF-A; (green) transfected with modified RNA encoding BMP-2; (red) co-transfected with modified RNA encoding VEGF-A and modified RNA encoding BMP-2.

FIGS. 8E-8H are bar graphs showing quantification of (E) ALP, (F) COL1, (G) OCN, (H) RunX2 protein levels measured by western blotting analysis at 4 weeks post-implantation of BMSCs harboring select modified RNA molecules; (black) transfected with no RNA; (blue) transfected with modified RNA encoding VEGF-A; (green) transfected with modified RNA encoding BMP-2; (red) co-transfected with modified RNA encoding VEGF-A and modified RNA encoding BMP-2.

FIGS. 8I-8L are bar graphs showing quantification of (I) ALP, (J) COL1, (K) OCN, (L) RunX2 expression measured by qRTPCR at 12 weeks post-implantation of BMSCs harboring select modified RNA molecules; (black) transfected with no RNA; (blue) transfected with modified RNA encoding VEGF-A; (green) transfected with modified RNA encoding BMP-2; (red) co-transfected with modified RNA encoding VEGF-A and modified RNA encoding BMP-2.

FIGS. 8M-8P are bar graphs showing quantification of (M) ALP, (N) COL1, (O) OCN, (P) RunX2 protein levels measured by western blotting analysis at 12 weeks post-implantation of BMSCs harboring select modified RNA molecules; (black) transfected with no RNA; (blue) transfected with modified RNA encoding VEGF-A; (green) transfected with modified RNA encoding BMP-2; (red) co-transfected with modified RNA encoding VEGF-A and modified RNA encoding BMP-2.

FIG. 9 is a schematic diagram of a BMSC-modRNA application for clinical bone repair.

FIGS. 10A-F are is a series of photos of microscope images (10A) and of growth plates (10B-D) that show osteoinduction of MSCs in the absence of dexamethasone (10A-D). Microscopic images of alkaline phosphatase staining of BMSCs at 7 days post transfections in different ratios of modRNA and recombinant proteins (10A). Gross appearance of ALP staining. Note appearance of ALP expression appears greatest in 3B1V and 1B1V groups (10B). Detection of mineralized matrix using alizarin red staining 14 days post transfection in the indicated modRNA or recombinant protein treatment groups (10C). Gross appearance of alizarin red staining (10D). Quantification of ALP activity at 7 days post treatment (10E) and alizarin red at 14 days-post transfection (10F).

FIGS. 10G-J are a series of bar graphs that show results of fold increase in gene expression of (10G) ALP, (10H) Collagen Type 1 (COL1), (10I) Osteocalcin (OCN), (10J) Runt-related transcription factor 2 (Runx2) at 7 days post transfection determined by qRT-PCR analysis when MSCs are transfected with modRNAs and recombinant proteins in the absence of dexamethasone.

FIG. 10K-O is a representation of a Western blot that shows representative western blot assessment (10K) and quantification of protein expression of ALP (10L), COL1 (10M), OCN (N) and Runx2 (10O) following the transfections of indicated modRNAs and recombinant proteins treatments in the absence of dexamethasone.

FIG. 11A is a series of representative images reconstructed from 3D μCT scans showing bone tissue regeneration at 4 weeks after treatment with Luc (control), 3B1V (BMP2+VEGF-A modRNA at a ratio of 3:1), 1B3V (ratio of 1:3), and rBV (recombinant human protein BMP and VEGF).

FIG. 11B is a bar graph that illustrates the results of testing as recited for FIG. 11A.

FIGS. 12A-D are a series of representations of microscopic images of hematoxylin and eosin (H&E) stained tissues of the cranial defect location at 4 weeks post-implantation of BMSCs harboring selected modified RNA molecules.

FIGS. 12E-H are a series of representative microscopic images of Masson's trichrome stained tissues of the cranial defect location at 4 weeks post-implantation of BMSCs harboring select modified RNA molecules.

FIGS. 13A-D are a series of representative photomicrographs of sectioned and Osteocalcin (OCN) stained bone tissue in the defect areas at 4 weeks post-treatment. The rectangular areas in FIGS. 13 A-D correspond to the magnified areas in 13A′-D′.

FIGS. 13E-H are a series of representative photomicrographs of sectioned and CD31 stained bone sections in the defect areas at 4 wks post-treatment. The rectangular areas in images 13E-H correspond to the magnified areas in images -13E′-H′.

FIGS. 13I and 13J are two bar graphs that show quantified assessment of OCN positive cells at the site of regenerating bone in indicated groups at 4 weeks post-treatment (13I), or the quantified assessment of CD31 labelling of the indicated treatment groups at 4 weeks post-treatment (13J).

FIGS. 14A-D are a series of bar graphs that show osteogenic gene related expression of (14A) ALP, (14B) COL1, (14C) OCN and (14D) Runx2 in regenerating bone at 4 weeks post-transfection quantified using qRT-PCR.

FIG. 14E is a representative Western blot used to perform quantification of osteogenic genes at the protein level following modRNA and recombinant protein treatments in regenerating bone at 4 weeks post-transfection.

FIGS. 14F-I are a series of bar graphs that show detection of protein levels for (14F) ALP, (14G) COL1, (14H) OCN and (14I) Runx2 in regenerating bone tissue using Western blot analysis at 4 weeks post-treatment.

### DETAILED DESCRIPTION

The present disclosure is based, at least in part, on the discovery that if one uses compositions of mesenchymal stem/stromal cells (MSCs), e.g., BMSCs, adipose/fat derived MSCs, peripheral blood derived MSCs, umbilical MSCs, or placental-derived MSCs, combined with solid or semisolid carriers, e.g., biodegradable carriers, wherein the MSCs harbor modified RNA molecules that encode VEGF polypeptides and BMP polypeptides, one can use those MSCs to deliver a controlled dosage of both of these proteins for a controlled length of time to provide an improved therapy to heal bone defects, including otherwise non-healing bone defects. The MSCs can be either used in autologous cell therapy in which the subject's (patient's) own cells are removed, engineered to add the modRNA, and then administered back into the same subject, or in allogeneic cell therapy, in which a composition of engineered MSCs from some other subject are administered to a subject.

Chemically modified mRNAs (“mod RNAs”) are a novel technology platform for restoring or enhancing expression of polypeptides (Zangi et al., Nat. Biotechnol., 31, 898-907 (2013); Carlsson et al., Mol. Ther. Methods Clin. Dev., 9, 330-346 (2018); Gan et al., Nat. Commun., 10, 871 (2019)). Unlike gene therapy, modified mRNAs deliver a transient “burst” of therapeutic protein expression while evading immune detection of the host cells. As described herein, this the onset and duration of this “burst” can be controlled.

The present disclosure relates to the therapeutic use of modRNA to repair bone defects, specifically the use of modRNA encoding two known factors, Bone Morphogenetic Protein, e.g., BMP-2 or BMP-7, and Vascular Endothelial Growth Factor, e.g., VEGF-A, which are key developmental and reparative regulators of bone growth (Wei et al., Tissue Eng. Pt. A, 24, 584-594 (2018); Hu et al., J. Clin. Invest., 126, 509-526 (2016)). We hypothesized that the co-expression of modRNAs coding for the human BMP-2 and VEGF-A gene could better accelerate bone healing and regeneration by enhancing osteogenesis through the simultaneous stimulation of angiogenesis. As described in more detail below, we first investigated the osteogenic potential of BMP-2 modRNA (modBMP2) together with the angiogenic potential of VEGF-A modRNA (modVEGF-A) in vitro using rat-derived BMSCs. BMSCs were transfected with modBMP2 and modVEGF-A at high efficiency. Following transfection of BMSCs, elevated levels of biologically active VEGF-A and BMP2 protein were detected for more than 5 days. The transfection of these modRNAs effectively induced osteogenic differentiation of BMSCs in vitro. In addition, the levels of transcripts associated with osteogenesis and angiogenesis were significantly increased for up to 2 weeks post-transfection.

We also investigated and confirmed the benefits of these methods in vivo by loading modBMP2 and modVEGF-A pre-treated-BMSCs into a collagen scaffold that we then implanted into calvarial defects in rats. As early as 4 weeks post-operation the animals receiving BMSCs pre-treated with modVEGF-A and modBMP2 showed significant bone healing. By 12 weeks post-operation the BMP2+VEGF-A modRNA treated group revealed significant levels of new bone formation. These experiments confirm the efficacy and synergistic effect of these methods to deliver low doses of BMP2 and VEGF-A using modRNAs to regenerate bone. These results provide insights to a novel, safe, efficient, and cost-effective approach for bone tissue regeneration.

### Modified RNA

Traditionally, the basic components of an mRNA molecule include at least a coding region, a 5′ untranslated region (UTR), a 3′ untranslated region (UTR), a 5′ cap, and a poly-A tail. Building on this wild type modular structure, the present disclosure expands the scope of functionality of traditional mRNA molecules by providing polynucleotides or primary RNA constructs which maintain a modular organization, but which comprise one or more structural and/or chemical modifications or alterations that impart useful properties to the polynucleotide. In some embodiments, modified RNAs comprise a modification to the 5′ cap, such as a 5′ diguanosine cap. In some embodiments, modified RNAs comprise a modification to a coding region. In some embodiments, modified RNAs comprise a modification to a 5′ UTR. In some embodiments, modified RNAs comprise a modification to a 3′ UTR. In other embodiments, modified RNAs comprise a modification to a poly-A tail. In some embodiments, modified RNAs comprise any combination of modifications to a coding region, 5′ cap, 5′ UTR, 3′ UTR, or poly-A tail.

In some embodiments, a modified RNA can optionally be treated with an alkaline phosphatase. Examples of modifications to RNA molecules are disclosed in U.S. Patent Application Publication No. 2014/0073687, U.S. Patent Application Publication No. 2015/0051268, and U.S. Pat. No. 9,061,059, the contents of which are all incorporated herein by reference in their entireties.

### Modified RNA Encoding BMP Polypeptide

As described in more detail in the Examples below, modified RNA molecules can be prepared that encode a BMP polypeptide, e.g., a human BMP, e.g., a BMP-2 polypeptide, which can be any one of a large family of BMP proteins that are known to play a central role in TGF-β-signaling and in modulating bone and cartilage biology. It will be appreciated by those of skill in the art that for any particular BMP gene there may exist one or more variants or isoforms.

A non-limiting example of the sequence of human BMP-2 polypeptide and the nucleic acid sequence that encodes this BMP-2 polypeptide, which can be used in accordance with the present disclosure, is available online at NM Ref. NM_001200.4 at the NCBI database. Other BMP-2 polypeptides and nucleic acid sequences are publicly available and can be used in the compositions and methods described herein. For example, the specific nucleic acids encoding BMP-2 and BMP-7 are known, and can be found in, e.g., van der Kraan et al., 2010, Osteoarthritis Cartilage. 2010 June; 18(6):735-41; and Lissenberg-Thunnissen et al., Int. Orthop. 2011 September; 35(9):1271-80.

Proper dosing of BMP polypeptide is important, because too much BMP polypeptide is known to cause unacceptable side effects. For example, while BMP polypeptides can promote bone formation, they also induce adverse effects, including cyst-like bone formation and significant soft tissue swelling. Several studies have shown these significant and unacceptable side effects. For example, one study evaluated multiple BMP2 doses in different rat models to determine dose-dependent effects (Zara et al., Tissue Eng., Volume 17, Numbers 9 and 10 (2011), DOI:10.1089/ten.tea.2010.0555). Results showed that only a mid-level dosage was able to fuse a bone defect without adverse effects (30 mg/mL, total dose 2.25 mg in 75 mg total volume), while a lower dose was ineffective, and a higher concentration range of BMP2 (150, 300, and 600 mg/mL, total dose 11.25, 22.5, and 45 mg in 75 mg total volume) was able to fuse the defect, but with formation of cyst-like bony shells filled with histologically confirmed adipose tissue. In addition, compared to control, 4 mg/mL BMP-2 also caused significant tissue inflammatory infiltrates and exudates in a femoral onlay model that was accompanied by increased numbers of osteoclast-like cells at 3, 7, and 14 days. Overall, the study shows that BMP-2 side effects of cyst-like bone and soft tissue swelling are caused when using high BMP-2 concentrations approaching the typical human dose of 1500 mg/mL

On the other hand, the present disclosure provides controlled concentrations of BMP, e.g., BMP-2, expression that provide effect healing of bone defects, but without the side effects described in the prior art. As described in more detail below, in the examples, modified mRNA in vivo expression kinetics are highly favorable over recombinant protein and adeno-viral associated gene delivery. Advantages of modified RNA include, but are not limited to, dosable and controlled protein levels, fast delivery, negligible immune reactions, and a transient signal.

### Modified RNA Encoding VEGF Polypeptide

As described in more detail in the Examples below, modified RNA molecules can be prepared that encode a VEGF polypeptide, e.g., a human VEGF, e.g., a VEGF-A polypeptide, which can be any one of a large family of VEGF proteins that play a central role in the control of cardiovascular physiological function (Holmes D. I. et al, Genome Biol, (2005)6(2):209). It will be appreciated by those of skill in the art that for any particular VEGF gene there may exist one or more variants or isoforms.

Non-limiting examples of the VEGF-A polypeptides that can be used in accordance with the present disclosure are listed in Table 1. These polypeptides and the nucleic acid molecules that encode them are publically available online at the NCBI database. Other VEGF polypeptides and nucleic acid sequences are publicly available and can be used in the compositions and methods described herein.

TABLE 1
Homo sapiens VEGF-A mRNA Isoforms

| Description | NM Ref |
|---|---|
| Homo sapiens VEGF-A, transcript variant | NM_001025366.3 |
| 1, mRNA | |
| Homo sapiens VEGF-A, transcript variant | NM_003376.6 |
| 2, mRNA | |
| Homo sapiens VEGF-A, transcript variant | NM_001025367.3 |
| 3, mRNA | |
| Homo sapiens VEGF-A, transcript variant | NM_001025368.3 |
| 4, mRNA | |
| Homo sapiens VEGF-A, transcript variant | NM_001025369.3 |
| 5, mRNA | |
| Homo sapiens VEGF-A, transcript variant | NM_001025370.3 |
| 6, mRNA | |
| Homo sapiens VEGF-A, transcript variant | NM_001033756.3 |
| 7, mRNA | |
| Homo sapiens VEGF-A, transcript variant | NM_001171622.2 |
| 8, mRNA | |
| Homo sapiens VEGF-A, transcript variant | NM_001204385.2 |
| 9, mRNA | |
| Homo sapiens VEGF-A, transcript variant | NM_001287044.2 |
| 10, mRNA | |

### Mesenchymal Stem/Stromal Cells

Mesenchymal Stem/Stromal Cells (SMCs) can be obtained from various sources, and can be obtained directly from the subject, engineered to harbor modRNA (and optionally cultured to increase the cell population, either before or after the modRNA is added), and then administered back into the same subject (autologous cell therapy) or the MSCs can be obtained from a first subject, engineered to add the modRNA (and optionally cultured to increase the cell population, either before or after the modRNA is added), and then administered to some other subject (allogeneic cell therapy), e.g., after storage, and thus can be available to treat a subject much more rapidly than using autologous cell therapy.

One example of MSCs are bone marrow stem cells (BMSCs), which can be directly isolated from subjects for use in autologous or allogenic cell therapies. U.S. Patent Application US20080279828A1 discloses methods of mobilization of bone marrow stem cells into the peripheral blood of a donor for harvesting the bone marrow stem cells, and is incorporated herein by reference in its entirety. The method comprises administering to the donor an effective amount of at least one copper chelate, to thereby expand the bone marrow stem cells in vivo, while at the same time reversibly inhibiting differentiation of the bone marrow stem cells; and harvesting the bone marrow stem cells by leukopheresis.

Alternatively, cells that differentiate into BMSCs (“BMSC precursors”) can be isolated from subjects and then exposed to one or more chemical or biological agents to differentiate into BMSCs in culture. U.S. Pat. No. 5,486,359 describes the isolation of human mesenchymal stem cells, which can differentiate into more than one tissue type (e.g. bone, cartilage, muscle, or marrow stroma) and a method for isolating, purifying, and culturally expanding human mesenchymal stem cells.

Additional sources of MSCs from adult niches including adipose/fat-derived MSCs and peripheral blood derived MSCs. In addition, MSCs from the pre/neo-natal environment can be used in the methods described herein, including umbilical and placental-derived MSCs. Human umbilical cord and placenta-derived MSCs, as well as peripheral blood derived MSCs can be isolated from patients using methods known in the art, e.g., through a combination of tissue explant cultures and/or by gradient density separation through centrifugation (Beeravolu et al., J. Vis. Exp., 122 (2017); Chong et al., J Orthop Res., 30(4):634-42 (2012). For the isolation of adipose/fat-derived MSCs, the cells can first be isolated using for example, methods involving liposuction and resection (Schneider et al., Eur. J. Med. Res., 22(1):17 (2017). Although some functional diversity exists within mesenchymal stromal cells derived from different patients and/or different tissue sources, for mesenchymal stem/stromal cells to maintain their identity they should possess three functional attributes: 1) self-renewal potential; 2) ability to grow on plastics; and 3) ability to differentiate into three major cell types including osteoblast (bone), chondrocyte (cartilage) and adipocyte (fat). Additionally, regardless of the source of MSCs, the MSCs should have differentiation markers such as CD73, CD90 and the lack of CD14, CD34, and CD45 (Ullah et al., Biosci. Rep., 35(2) (Apr. 28, 2015); Fitzsimmons et al., Stem Cells Int. 2018: 8031718 (2018)).

### Preparation of Compositions of MSCs Harboring Modified RNA Encoding BMP Polypeptide and Modified RNA Encoding VEGF Polypeptide

Modified RNA encoding BMP polypeptide and/or modified RNA encoding VEGF polypeptide can be delivered into MSCs by methods known in the art. Methods include, but are not limited to, electroporation, transfection (e.g., methods using cationic-lipid transfection reagents), and lipid nanoparticle encapsulating the modified RNAs.

In particular, one can prepare the MSCs described herein using the following steps.

Delivering modRNA into MSCs BMSCs are grown in culture—e.g., seeded at 20-30×10⁴ cells/well in 6-well plates or flasks and seeded at approximately 4,000-6,000 cells per cm² in 0.2-0.4 mL/cm² media. For example, MSCs grown in T-75 flasks are generally seeded at 300,000 cells/flask in 15 mL of media. ModRNA complexes are formed with a cationic-lipid transfection reagent and incubated with BMSCs in culture. For example, the modRNA complexes can be—e.g., formed by using 2.5 μl Lipofectamine® MessengerMAX™ Reagent (RNAiMax Reagent and Lipofectamine 2,000 Reagent and 3,000 Reagent are also effective) per 1 μg modRNA. Calculations are performed to transfect BMSCs at a dose of 10 pg/cell modRNA (i.e., Luciferase, GFP, mCherry, hBMP-2 or VEGF-A). Ratios of modified mRNA to cells can range from 1 pg/cell to 100 pg/cell.

In some embodiment, the concentration of modified mRNA can be increased or decreased from 1 μg/μl to enable a lower or higher dose and thus, different levels of the modified mRNA in the cells. In some embodiments different ratios of the modRNA transcripts can be used, e.g., other than 1:1 ratios. For example, ratios of 2:1, 3:1, 4:1, and 5:1 of the modRNA encoding VEGF and the modRNA encoding BMP, as well as ratios of these same polypeptides of 1:5, 1:4, 1:3, and 1:2 can be envisioned depending on the specific bone defect and subject.

In some embodiments, the cells can be single-transfected in two groups to obtain specific dosages of mRNA transcripts in each group, and then combined at a later time, or the transfections can be tailored so that the same cells harbor different doses/concentrations of the mRNA transcripts.

Transfection Methods

MSCs can be grown in culture and transfected, e.g., electroporated, with specific doses of modRNA. In some embodiments, the human MSCs, e.g., hBMSCs, are transfected with modRNA using Nucleofector™ 2b and the hMSC Nucleofector™ kit (Lonza) according to the manufacturer's instructions. In brief, cells are resuspended in 100 μL Nucleofector™ solution, mixed with modified mRNA (e.g., at 100 ng-100 μg per 1 million cells), transferred to a cuvette, and electroporated using program U-23 of the Nucleofector™ device. Nucleofected samples can be placed in pre-warmed medium to recover or resuspended in a low glucose DMEM solution supplemented with FBS and Pen/strep (such as Lonza™ hMSC-GM) and 10% DMSO, and frozen at −80° C. (and can be stored in liquid nitrogen tanks at −180° C.) until further use.

Fabrication and Functionalization of Solid and Semi-Solid Carriers

Carriers can be made of one or more of the following bio-inert or bioactive materials that may or may not be biodegradable and/or bioabsorbable: bioceramics, bioactive glasses, polymer components/nano fibers (e.g., polyfumarates, polycaprolactone, polylactic acid (PLA), polyglycolic acid (PGA), copolymers of PLA and PGA (PLGA), nanomaterials (e.g., nanotubes), hydroxyapatite (HA) (in the form of crystals or other), collagen fibers/sponges, collagen molecules, non-toxic lipid nanoparticles, and/or gel/silica-based hydrogels. Various other pharmaceutically acceptable polymers can also be used, as long as they can be used to deliver the compositions described herein to a specific site of a bone defect, and maintain all or at least some of the compositions at that site for a sufficient duration, e.g., 1, 2, 3, 4, 5, 6, or 7, or more days, 1, 2, 3, 4, 5, 6, 7, 8, 9, 10, 11, 12, or more weeks, or for 1, 2, 3, 4, 5, 6 or more months. The carriers can be designed to biodegrade at a known rate to help control the duration of treatment with the engineered MSCs.

In one example, collagen sponges can be prepared, for example, by partially dissociating in 0.1 mol/L acetic acid and frozen at −80° C., then freeze-dried at −50° C. under a vacuum, e.g., for a sufficient time, e.g., 24 hours, subsequently cross-linked, e.g., with 25 mM EDC and 25 mM NHS with 95% ethanol solution for a sufficient time, e.g., 24 hours, thoroughly washed with a washing solution, such as 5 wt % glycine solution and distilled water, e.g., three times, and freeze-dried a second time at −50° C. to obtain a collagen scaffold.

Preparation of modRNA-BMSCs/Scaffold Constructs and Surgical Procedures

The modRNA-transfected cells can be trypsinized after 4 hours of modified mRNA transfection, centrifuged, and resuspended in osteoblast inducing conditional medium. The cells (5×10⁶ cells/ml) can be evenly seeded into cross-linked collagen scaffolds, e.g., 50 μl/scaffold, thus 2.5×10⁵ cells/scaffold. Osteogenic medium can be added after 4 hour incubation at 37° C., and the BMSCs/scaffold constructs are incubated overnight prior to implantation. The absorbable collagen sponges are biodegradable. The inoculation of the mRNA-loaded BMSCs in biomaterials help control growth factor release locally at the site of a bone defect, as well as enhance secretion and expression over longer periods of time until the support material is completely degraded.

When made and used according to the methods described herein, the new MSC compositions provide a synergistic effect from the combination of VEGF-A and BMP-2 polypeptides, because as shown in FIGS. 5A-5G as described in further detail in the Examples below, the new compositions provide more robust and effective bone regeneration than using twice the concentration of either VEGF-A or BMP-2 polypeptides delivered alone. In other words, the new compositions deliver a safe and effective low dose of VEGF-A and BMP-2 to the bone defect that is more effective than much higher doses (twice the concentration) of either polypeptide delivered alone.

Methods of Treatment

The methods described herein include methods for the treatment of bone defects, such as non-healing bone defects in subjects, such as human subjects. Generally, the methods include administering a therapeutically effective amount of MSCs harboring modRNA encoding BMP-2 polypeptide and modRNA encoding VEGF-A polypeptide as described herein, to a subject who is in need of, or who has been determined to be in need of, such treatment.

As used in this context, to “treat” means to ameliorate or partially or fully heal the bone defect. Often, treatment with the compositions described herein—MSCs harboring modRNA encoding BMP-2 polypeptide and modRNA encoding VEGF-A polypeptide-results in partial or complete healing of the bone defect.

In particular, one can treat a subject with the compositions described herein by administering, e.g., locally administering, the compositions described herein to the site of a bone defect. Such administration can include injection to a small site, or a surgical procedure to make the site of the bone defect sufficiently accessible so that the compositions can be applied directly to the bone defect. For example, cells harboring modified mRNAs can be delivered via systemic or local injection and/or infusion through vascular catheterization procedures. In addition, the cells can be engineered to migrate to the areas of bone defect, using known techniques. If the BMSCs harboring modRNA are included in biomaterials to form the compositions described herein, delivery routes to bone may require surgical procedures including, but not limited to, core decompression, which involves drilling into the bone, often in diseased/injured areas for cell/bone grafting (Steinberg M E, Can J Surg., 1995 February; 38 Suppl., 1:S18-24).

Therapeutically effective amounts of the new compositions are determined by the total number of cells administered and by the concentration of modRNA in each cell. The correct dose of cells harboring the mRNA will depend on multiple parameters including degree of injury, the size/weight of the subject, and the overall health of the subject. Based on data acquired in rat model systems as described herein, and on data regarding FTIH studies (Gan et al., Nat. Commun., 10(1):871 (2019)), the physiological dose range needed to repair a bone defect in human subjects would be in the range of about, e.g., about 2 μg to about 12 mg of modified mRNA, e.g., about 5 μg to about 1.0 mg, or about 10 μg to about 500 μg, and this dose depends in large part on the size of the bone defect to be repaired.

As discussed below and illustrated in FIG. 5, a composition of about 200,000 cells containing a dose of 2 μg modRNA (10 pg/cell total VEGF and BMP mRNA, e.g., 5 pg each) was used to almost entirely fill a defect of about 5 mm in diameter at the beginning of a 12 week period. In other embodiments, the same defect could be filled with a composition including 400,000-500,000 cells rather than 200,000 cells. The dose per cell is unchanged, but using more cells per composition results in an increased payload of secreted proteins. Clinically, burr holes range in size from 10 mm-50 mm in diameter. So, for example, to treat a bone defect/burr hole that is roughly 10 mm in diameter, e.g., in the skull, that is about 4-8 mm thick, one should administer approximately 1 million MSCs with an mRNA dose of 10 μg/cell (i.e., 10 modRNA).

The modRNA can be administered as a single dose or as multiple doses that add up to the total dose over several administrations.

The dose of the BMP polypeptides expressed by the MSCs in the new compositions described herein is carefully controlled to avoid delivery of too high a dose of the BMP polypeptides. For example, a high dose rhBMP2 (40 mg) product called AMPLIFY® by Medtronic was administered to patients with degenerative lumbar disease and at 2 year follow-up patients had over a 300% increase in cancer cases in their treated patients (Panel OaRDA P050u36, 2010; Carragee et al., J. Bone Joint Surg. Am., 95:1537-45 (2013); Dimar et al., J. Bone Joint Surg. Am., 91:1377-1386 (2009)). On the other hand the INFUSE clinical trial used 6 and 12 mg doses and have reported a lower cancer risk (Simmonds et al., Ann. Intern. Med., 158(12):877-889 (2013)). See also, Epstein, “Complications due to the use of BMP/INFUSE in spine surgery: The evidence continues to mount,” Surg. Neurol. Int., 4(supp15):5343-5352 (2013).

Without wishing to be bound to a particular theory, a useful dose of the BMP component delivered by the new compositions described herein is in the range of about 2 μg-12 mg, e.g., 2, 5, 10, 25, 50, 100, 200, 300, 400, 500, 600, 700, 800, 900, or 1000 pg, ng, or μg, or 1, 2, 3, 4, 5, 6, 7, 8, 9, 10, 11, or 12, or more mg. Similar levels of VEGF polypeptides should also be delivered. The particular “dose” is also determined by the amount of modRNA molecules in the cells. A useful dosage range of the cells harboring the modified mRNAs is about 1 pg/cell to 100 ng/cell, e.g., 1, 2, 3, 4, 5, 6, 7, 8, 9, 10, 20, 30, 40, 50, 60, 70, 80, 90, or 100 pg/cell, with a total useful range of between 1 pg/cell to 100 ng/cell, e.g., 200, 300, 400, 500, 600, 700, 800, 900, or 1000 pg/cell. Cell number determines final dose of modified mRNAs as it is an additive effect. Thus, the cell number range administered to the site of the bone defect should be in the range of about 20×10⁴ to about 120×10⁶ cells, e.g., 20×10⁵, 1×10⁶, 20×10⁶, 50×10⁶, or 100×10⁶.

The duration of the burst of polypeptide or protein is determined by a combination of both cell number and the level of modified mRNA transcripts that are introduced within the cells. The burst of secreted polypeptides or proteins should be selected to last between 2 and 5 days, but due to the half-life of the newly accumulated protein, they may be detected for up to 2 weeks.

### EXAMPLES

The invention is further described in the following examples, which do not limit the scope of the invention described in the claims.

### Materials

Dulbecco's modified eagle medium (DMEM) and fetal bovine serum (FBS) were purchased from Gibco (Thermo Fisher Scientific, USA). Trypsin-EDTA (0.25%, 1× solution), Dulbecco's phosphate buffered saline (DPBS), and antibiotics (100 U/mL penicillin G and 0.1 mg/mL streptomycin) were purchased from Gibco® (Invitrogen™ Grand Island, N.Y.). Cell Counting Kit-8 (CCK-8) was obtained from Dojindo Laboratories (Dojindo, Kumamoto, Japan). Alizarin Red S was purchased from Solarbio®Life Sciences, Alkaline phosphatase (ALP) staining kit (BCIP/NBT Alkaline Phosphatase Color Development Kit) was purchased from Beyotime Institute of Biotechnology. Collagen was obtained from Sichuan Mingrang Tech., China, and collagen cross-linking agent N-(3-(dimethylamino)-propyl)-N-Ethylcarbodiimide hydrochloride crystalline (EDC) and N-Hydroxysulfosuccinimide sodium salt (NHS) were purchased from Aladdin Industrial Corporation, Shanghai, China. All materials were utilized as received, without any further purification.

### Example 1—Demonstration that Bone Marrow Stem Cells (BMSCs) Transiently Transfected with Modified RNA (modRNA) Exhibit Expression with Low Cytotoxicity

To determine BMSCs can be co-transfected with two modRNAs with low cytotoxicity, BMSCs were transiently co-transfected with modRNAs encoding green fluorescent protein (GFP) and modRNAs encoding mCherry. Expression of the fluorescent proteins was measured along with cytotoxicity by a cell counting and proliferation assays.

### Methods

Generation of Chemically Modified mRNA (modRNA) Encoding GFP, mCherry, Luciferase, Human BMP2 (hBMP-2), and Human VEGF-A Complexes

MRNA was synthesized in vitro using T7 RNA polymerase-mediated transcription from a linearized DNA template, which incorporates the 5′ UTR, 3′UTR, and a poly-A tail as previously described (Richner, J. M., et al. Cell 169, 176 (2017)). RNA was purified using Ambion MEGA clear spin columns and then treated with Antarctic Phosphatase (New England Biolabs) for 30 min at 37° C. to remove residual 5′-phosphates. Treated RNA was re-purified and quantified by Nanodrop (Thermo Scientific). After purification, modRNA was resuspended in 10 mM Tris HCl, 1 mM EDTA at 1 μg/μl for use. In mRNA, uridine was fully replaced by N1-methylpseudouridine. GFP, mCherry, firefly luciferase, and human VEGF-A (165) ORF sequences were the same as previously described (Zangi, L., et al. Nat Biotechnol 31, 898-907 (2013)).

The open reading frame sequence for human BMP-2 modRNA was:

(SEQ ID NO: 1)
5′-ATGGTGGCCGGGACCCGCTGTCTTCTAGCGTTGCTGCTTCCCCAGGT
CCTCCTGGGCGGCGCGGCTGGCCTCGTTCCGGAGCTGGGCCGCAGGAAGT
TCGCGGCGGCGTCGTCGGGCCGCCCCTCATCCCAGCCCTCTGACGAGGTC
CTGAGCGAGTTCGAGTTGCGGCTGCTCAGCATGTTCGGCCTGAAACAGAG
ACCCACCCCCAGCAGGGACGCCGTGGTGCCCCCCTACATGCTAGACCTGT
ATCGCAGGCACTCAGGTCAGCCGGGCTCACCCGCCCCAGACCACCGGTTG
GAGAGGGCAGCCAGCCGAGCCAACACTGTGCGCAGCTTCCACCATGAAGA
ATCTTTGGAAGAACTACCAGAAACGAGTGGGAAAACAACCCGGAGATTCT
TCTTTAATTTAAGTTCTATCCCCACGGAGGAGTTTATCACCTCAGCAGAG
CTTCAGGTTTTCCGAGAACAGATGCAAGATGCTTTAGGAAACAATAGCAG
TTTCCATCACCGAATTAATATTTATGAAATCATAAAACCTGCAACAGCCA
ACTCGAAATTCCCCGTGACCAGACTTTTGGACACCAGGTTGGTGAATCAG
AATGCAAGCAGGTGGGAAAGTTTTGATGTCACCCCCGCTGTGATGCGGTG
GACTGCACAGGGACACGCCAACCATGGATTCGTGGTGGAAGTGGCCCACT
TGGAGGAGAAACAAGGTGTCTCCAAGAGACATGTTAGGATAAGCAGGTCT
TTGCACCAAGATGAACACAGCTGGTCACAGATAAGGCCATTGCTAGTAAC
TTTTGGCCATGATGGAAAAGGGCATCCTCTCCACAAAAGAGAAAAACGTC
AAGCCAAACACAAACAGCGGAAACGCCTTAAGTCCAGCTGTAAGAGACAC
CCTTTGTACGTGGACTTCAGTGACGTGGGGTGGAATGACTGGATTGTGGC
TCCCCCGGGGTATCACGCCTTTTACTGCCACGGAGAATGCCCTTTTCCTC
TGGCTGATCATCTGAACTCCACTAATCATGCCATTGTTCAGACGTTGGTC
AACTCTGTTAACTCTAAGATTCCTAAGGCATGCTGTGTCCCGACAGAACT
CAGTGCTATCTCGATGCTGTACCTTGACGAGAATGAAAAGGTTGTATTAA
AGAACTATCAGGACATGGTTGTGGAGGGTTGTGGGTGTCGCTAG-3′.

Isolation and Culture of Rat BMSCs

A cell isolation process was performed as previously described (Li et al., Cytotechnology, 65:323-334 (2013); Li et al., Biomaterials, 74:155-166 (2016)). Primary BMSCs were harvested from the limbs (ulnae, radii, femora and tibiae) of 1-week old male Sprague-Dawley rats (Shanghai SLAC Experimental Animal Center, Shanghai, People's Republic of China) and the bone marrow was flushed by 26 G needle attached to a 1 ml disposable aseptic syringe containing DMEM medium. The cells were filtered through a 70 μm filter mesh to remove bone spicules and cell clumps, resuspended in complete low glucose DMEM medium containing 10% FBS, Gibco, 100 IU/ml penicillin and 100 IU/ml streptomycin, incubated at 37° C. with 5% CO₂ in a humidified chamber and were passaged 2-3 times for down-stream experimentation. BMSCs that were isolated following the described protocols above were characterized by flow cytometry and multi-lineage differentiation assays using known techniques. To enhance osteogenic differentiation, BMSCs were cultured with osteogenic medium following modRNA transfection. The osteogenic differentiation medium (i.e. 10% FBS, 10 mM β-glycerophosphate, 0.05 mM ascorbic acid, and 10 nM dexamethasone) was prepared in advance. The hBMP-2, VEGF-A and hBMP-2+VEGF-A modRNA was transfected into the BMSCs as previously described. The medium was exchanged with osteogenic medium 4 h post-transfection for all groups. Transfected cells were sustained under osteogenic conditions for up to 14 days and the medium was changed every 2-3 days. Non-transfected cells, served as a control group, were cultured under the same terms and conditions.

ModRNA Preparation and BMSCs Transfection

BMSCs were transfected using Lipofectamine® MessengerMAX™ Reagent (Invitrogen, Life Technologies, USA) according to the manufacturer's instructions. Briefly, BMSCs were seeded at 20×10⁴ cells/well in 6-well plates. After incubation of 24 hours, cell culture medium was replaced with fresh reduced-serum medium Opti-MEM (Gibco, Life Technologies, USA). ModRNA complexes were formed by using 2.5 μl Lipofectamine® MessengerMAX™ Reagent per 1 μg modRNA. Calculations were performed in order to transfect BMSCs at a dose of 10 pg/cell modRNA (i.e. Luciferase, GFP, mCherry, hBMP-2 or VEGF-A). Briefly, in tube A, 2 μl of modRNA (1 μg/μl) was added to every 48 μl Opti-MEM and incubated for 5 min; in tube B, 5 μl Lipofectamine was added to every 45 μl Opti-MEM and incubated for 5 min. Subsequently, tube A and B were mixed and incubated at room temperature for 15 min. Next, old medium was removed from the cells and 1.5 ml fresh Opti-MEM was added to each well (Lui, K. O., et al. Cell Res 23, 1172-1186 (2013)). Finally, 100 μl of AB mixture was added to each well containing 2 μg modRNA/well. At 4 hours after transfection, medium was replaced with complete DMEM. The cells were further cultured under standard conditions for up to 7 days until analysis.

Verification of Translation from Co-Transfected Modified mRNAs in BMSCs

To confirm expression of two simultaneous modRNA transfections in BMSCs, two constructs—a GFP modRNA and an mCherry modRNA—were transfected singly or in combination. BMSCs were divided into 1 of 3 treatment groups: 1) GFP modRNA transfection only; 2) mCherry modRNA transfection only; 3) GFP modRNA combined with mCherry modRNA. At 24 hours post-transfection, cells were analyzed using fluorescence microscopy.

Statistical Analysis

The statistical analyses were performed using GraphPadμ Prism Version 7.00 (GraphPad Software, CA, USA). Results were expressed as mean±standard deviation (SD) and unpaired Student's t-test was employed to analyze the differences between the untransfected and transfected group. One-way ANOVA were performed to analyze comparison of multiple groups. ANOVA analyses were corrected for multiple comparison by Tukey (Gaussian distribution) and Kruskal-Wallis or Dunn's (non-Gaussian distribution) tests. Experiments were performed in triplicate and differences were considered statistically significant at the levels of *p<0.05, **p<0.01 and ***p<0.001.p<0.05 represents statistical significance.

### Results

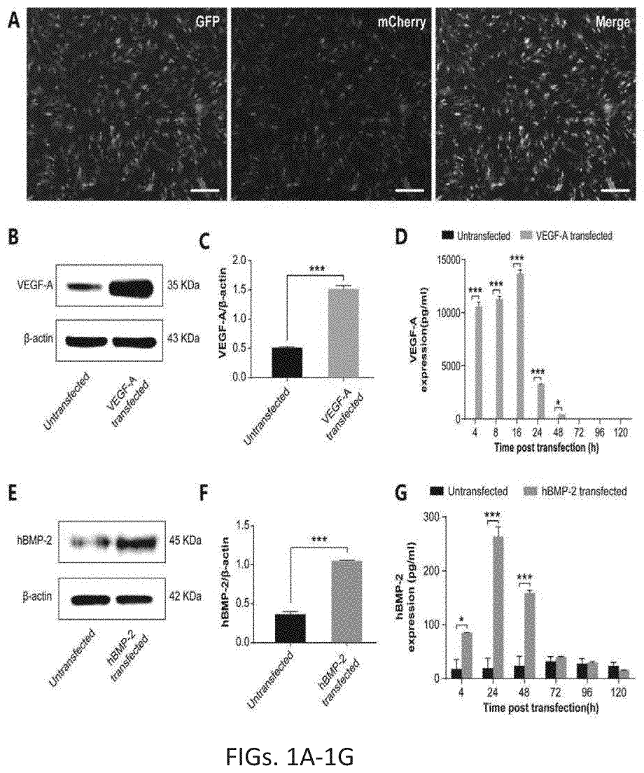

The mRNA molecule encoding GFP and mRNA molecule encoding mCherry were modified by the full replacement of uridine-5′-triphosphate with N¹-methylpseudo-uridine-5′-triphosphate (m1ψ). Co-transfections were performed into BMSCs of reporter modRNAs; a GFP and an mCherry modRNA construct. At 24 hours post-transfection, expression kinetics of the co-transfected modified mRNAs in BMSCs were analyzed using fluorescence microscopy and photomicrographic images are represented in FIG. 1A. The total proportion of cells appeared positive and the fluorescence over-lapped evenly between the GFP and mCherry channels. The calculated modRNA dosage equates to 10 pg/cell in total, 5 pg/cell for each GFP and mCherry modRNA respectively.

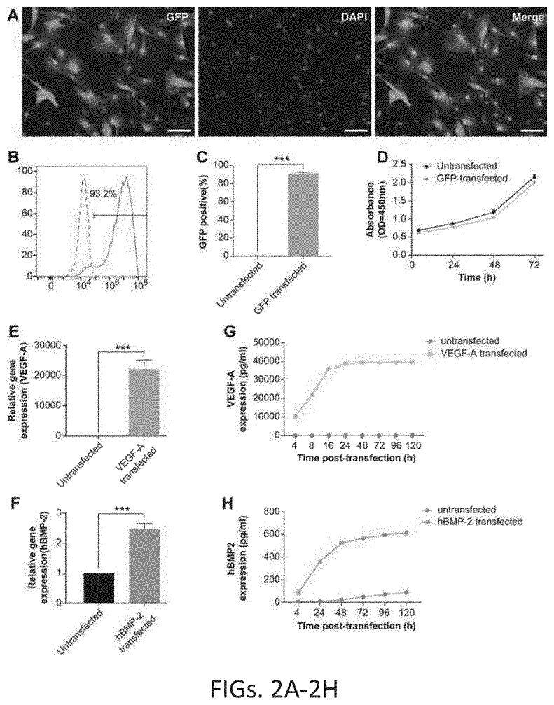

GFP expression was visualized within several hours after transfection (FIG. 2A), and flow cytometry analysis revealed that >90% of BMSCs expressed GFP protein 24h post-transfection (FIGS. 2B-2C). Furthermore, the cytotoxicity of GFP modRNA complexes (containing 1 μg of modRNA), was evaluated over 48 h in BMSCs using a CCK-8 assay (obtained from Dojindo Laboratories (Dojindo, Kumamoto, Japan) and performed according to manufacturer's instructions). As shown in FIG. 2D, the proliferation for modRNA transfected BMSCs was similar to the untransfected group, suggesting that modRNA-polyplexes have no significant influence on cell proliferation, a vital indicator of cell health (Adan, A., et al. Curr Pharm Biotechnol 17, 1213-1221 (2016)).

### Example 2—Demonstration that BMSCs Co-Transfected with BMP-2 modRNA and VEGF-A modRNA Express and Secrete High Levels of BMP-2 and VEGF-A Proteins

Towards assessing the co-transfection of BMSCs with two modRNAs—one encoding human BMP-2 and the other encoding human VEGF-A—BMSCs isolated from rats were co-transfected with said modRNAs and the modRNA expression and levels of proteins encoding by the modRNAs were evaluated by quantitative real-time PCR (qRT-PCR) and by western blotting, respectively. Additionally, the propensity of the BMSCs co-transfected with BMP-2 modRNA and VEGF-A modRNA for osteogenic differentiation was inferred by measuring expression of select genes with roles is osteogenesis and markers of osteogenic differentiation—specifically, alkaline phosphatase activity and calcium deposition. Finally, angiogenesis potential of the BMSCs co-transfected with BMP-2 modRNA and VEGF-A modRNA was assessed using an endothelial tube formation assay.

### Methods

BMSCs were prepared as described in Example 1. BSMCs were co-transfected with VEGF-A modRNA and BMP2 modRNA as described in Example 1.

Expression and Quantification of VEGF-A and hBMP-2 using Western Blotting

For western blot analysis, BMSCs were washed with PBS and lysed by protease inhibitor-containing RIPA buffer at 4° C. for 30 minutes. The lysates were centrifuged and samples were separated by sodium dodecyl sulfate-polyacrylamide gel electrophoresis (SDS-PAGE) and transferred onto a polyvinylidene difluoride (PVDF) membrane (Millipore, MA). The membrane was blocked with 5% bovine serum albumin (BSA) and probed with the primary antibodies from Abcam. The rat β-actin was used as internal control. The primary antibodies were rabbit anti-Rat VEGF and rabbit anti-Rat BMP-2. After rinsing, the membranes were incubated with horseradish peroxidase-conjugated secondary goat anti-rabbit IgG (1:1000, Beyotime) for 1 hour at room temperature. Then the target bands were acquired with an ECL Plus Western Blotting Detection System (GE Healthcare, IL, USA).

Quantification of BMP2 and VEGF-A Secretion by ELISA

To quantify the BMP2 and VEGF-A expression following BMSC transfection, the supernatant was sampled at different days post-transfection (dpt) for ELISA analysis using the human BMP-2 ELISA kit (Quantikine, R&D Systems, USA) and human VEGF ELISA kit (Quantikine, R&D Systems, USA), respectively, according to the manufacturer's instructions. The absorbance was measured at 450 nm. Wavelength correction was set at 570 nm. Experiments were performed in triplicates, and the protein content was determined using a standard curve (range: 0-1000 pg/ml BMP-2 and 0-1000 pg/ml VEGF).

Osteogenic Differentiation in Vitro

To enhance osteogenic differentiation, BMSCs were cultured with osteogenic medium following modRNA transfection. The osteogenic differentiation medium (i.e., 10% FBS, 10 mM β-glycerophosphate, 0.05 mM ascorbic acid, and 10 nM dexamethasone) was prepared in advance. hBMP-2, VEGF-A and hBMP-2+VEGF-A modRNA was transfected into the BMSCs as previously described (Insert Reference). The medium was exchanged with osteogenic medium 4 h post-transfection for all groups. Transfected cells were sustained under osteogenic conditions for up to 14 days and the medium was changed every 2-3 days. Non-transfected cells cultured under the same conditions served as a control group.

qRT-PCR and Western Blotting Analysis for Osteogenesis in Vitro

At 7 days post-osteogenic induction (DPOI) total RNA was extracted from transfected BMSCs using EZ-press RNA Purification Kit, according to the manufacturer's instructions as previously described. The osteo-related gene expression levels were determined by qRT-PCR analysis with a real-time thermal cycler using 2×SYBR Green PCR master mix with 1.5 μg of RNA. For qRT-PCR, primers specific for alkaline phosphatase (ALP), type I collagen (COL1), osteocalcin (OCN) and runt-related transcription factor 2 (RUNX2) genes (Table 2) were used.

TABLE 2
Rat Primers used in qRT-PCR Analysis

| | | Product | SEQ |
|---|---|---|---|
| Primers | Sequence (5′-3′) | size(bp) | ID NO: |
| GAPDH-F | GCCATCAACGACCCCTTCAT | 136 | 2 |
| GAPDH-R | AGATGGTGATGGGTTTCCCG | | 3 |
| ALP-F | CCTTAAGGGCCAGCTACACC | 100 | 4 |
| ALP-R | AGCGTTGGTGTTGTACGTCT | | 5 |
| COI-F | TGGTGAGACGTGGAAACCTG | 193 | 6 |
| COI-R | CTTGGGTCCCTCGACTCCTA | | 7 |
| OCN-F | GGTGGTGAATAGACTCCGGC | 117 | 8 |
| OCN-R | AGCTCGTCACAATTGGGGTT | | 9 |
| RUNX2-F | ACTACTCTGCCGAGCTACGA | 86 | 10 |
| RUNX2-R | GCTCCGGCCTACAAATCTCA | | 11 |

Alternatively, cell samples were harvested at 3 dpoi washed with PBS and lysed by protease inhibitor-containing RIPA buffer at 4° C. for 30 min, then followed by western blot. The rat β-actin was used as internal control. The primary antibodies were rabbit anti-Rat ALP, rabbit anti-Rat COL1, rabbit anti-Rat OCN, and rabbit anti-Rat RUNX2 (all from Abcam). The secondary antibody was HRP-conjugated goat anti-rabbit IgG (1:1000, Beyotime). Then the target bands were acquired with an ECL Plus Western Blotting Detection System (GE Healthcare, IL, USA).

Alkaline Phosphatase Activity Assay

Alkaline phosphatase activity (n=3) was assessed utilizing an ALP assay kit according to the manufacturer's protocol, as described previously (Yao, Q., et al. Electro-responsive BMP2 Release and Enhancement of Osteogenic Differentiation. 9, 39962-39970 (2017); Fahimipour, F., et al. Dent Mater 33, 1205-1216 (2017)). After osteogenic induction culture for 7 days, the cell layers were washed gently with cold PBS and digested with 0.25% trypsin-EDTA for 1 minute. The cell lysates were centrifuged at 1500 rpm for 5 min at 4° C. Then cell precipitate was lysed in 200 μl of 0.2% Triton X-100 for 30 min. Next, 30 μl of the supernatant was mixed with 150 μl of the working solution following the manufacturer's protocol (Nanjing Jiancheng Bioengineering Institute, Nanjing, China). The substrate of ALP, namely, the resultant of p-nitrophenol from p-nitrophenylphosphate, was evaluated by measuring the absorbance at 520 nm with a microplate reader (SpectraMax® i3x, Molecular Devices, Sunnyvale, Calif., USA). The ALP activity was calculated by the ratio of experimental samples to standard and was expressed by milligrams of p-nitrophenol produced per minute per gram of protein (king unit/gprot). Furthermore, BMSCs were subject to alkaline phosphatase staining at 7 dpoi as described previously (Zhou, G., et al. Int. J. Nanomedicine, 12, 7577-7588 (2017)).

Alizarin Red Staining and Quantification

To detect mineralization, BMSCs were subject to Alizarin red staining at 14 dpoi as described previously (Liao, Y. H., et al. Biomaterials 35, 4901-4910 (2014)). After removing the medium, the cells were washed twice with PBS and fixed with 4% paraformaldehyde for 30 minutes. Then cells were rinsed with PBS twice, and stained with 0.2% Alizarin Red S solution (pH 4.2) for 5 minutes, followed by three times PBS wash extensively to remove unspecific staining or verisimilar sediments. Calcified nodules are stained as red spots and were photographed by a microscope. To quantify the calcium deposition, after washing with dH₂O, the stain was solubilized with 10% cetylpyridinium chloride monohydrate (CPC, Sigma-Aldrich) in 0.1M PBS (pH 7.0) by shaking for 15 min and the absorbance was read at 570 nm (Lopa, S., et al. Eur Cell Mater 27, 298-311 (2014)). The calcium deposition was expressed as optical density (OD).

### Results

RNA was isolated from BMSCs 24 hours post-transfection of BMP2 and VEGF-A modRNA. Consequently, mRNA transcript levels were quantified by qRT-PCR. Compared to untransfected BMSCs, the modRNA transfections resulted in significantly higher levels of mRNA inside the cells (***p<0.001), (FIGS. 2E, 2F).

To evaluate BMP2 and VEGF-A target protein expression levels after lipofectamine transfection, protein levels of cell lysates at 24 hours post-transfection were quantified. The protein levels were significantly increased above basal levels compared to untransfected groups (FIG. 1B-C, E-F) respectively (***p<0.001). In order to capture and quantify secreted protein levels of BMP2 and VEGF-A, cell culture supernatants were collected at different time points after transfection. Quantification of BMP2 and VEGF-A protein using ELISA (R&D Systems, MN, USA) revealed significant increases in levels of cumulative protein secretion for at least the first 2 days in both groups (***p<0.001), (FIGS. 2G, 2H). According to a time course evaluation, maximum VEGF-A expression was observed 16 hours post transfection, while maximum BMP2 expression was observed at 24 hours post-transfection, (FIGS. 1D, 1G).

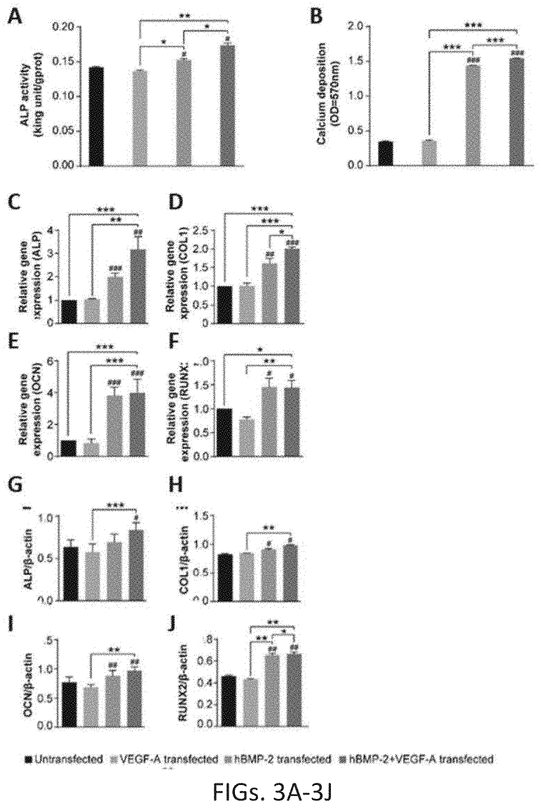

Alkaline phosphatase (ALP) is an early in vitro indicator of osteogenic differentiation, therefore increased ALP activity in our modRNA-transfected BMSCs would be a desirable feature. The ALP activity of BMSCs was assessed following transfection of modBMP2 alone, modVEGF alone, or as a combinatorial treatment together (modBMP2+modVEGF-A). ALP expression was observed in the modBMP2 and BMP2+VEGF-A modRNA treatment groups at 7 days post transfection, but not in the modVEGF-A transfected group. In addition, quantitative analysis of ALP activity through a colorimetric assay revealed a significant increase in contrast to untransfected BMSCs (FIG. 3A, *p<0.05, **p<0.01).

Calcium deposition is a late stage marker for osteogenic differentiation (Birmingham et al., Eur. Cells Mater., 23, 13-27 (2012)). Microscopic analysis of alizarin red staining at 14 days post-transfection revealed an increased number of calcium nodules in the modBMP2 group and a more pronounced effect in the BMP2+VEGF-A modRNA treatment group. Mineralization did not appear to occur in the modVEGF-A treatment group as alizarin red staining levels were consistent with levels seen in the control group. Quantitative assessment of the alizarin red staining (FIG. 3B) confirmed these results (p<0.001).

The expression of osteogenic related genes was quantified at 7 days post osteogenic induction (dpoi) by qRT-PCR, as shown in FIG. 3C-3F. Expression of ALP, COL1, and OCN were all significantly increased by BMP2+VEGF-A modRNA treatment (p<0.001, N=3). A similar effect was seen on Runx2, but to a lesser extent (P<0.05, N=3). Subsequently, western blot analysis was performed to evaluate quantitative changes in protein levels of these osteogenic-related genes following modRNA treatment. BMSCs treated with modBMP2 exerted increased protein levels of osteogenic genes over controls. Further, as seen with mRNA/gene expression levels, modVEGF-A treatment alone did not elicit increased protein levels, however the protein levels of ALP, COL1, OCN, and Runx2 were further increased in BMSCs treated with BMP2+VEGF-A modRNA (P<0.05, N=3), as shown in FIGS. 3G-3J.

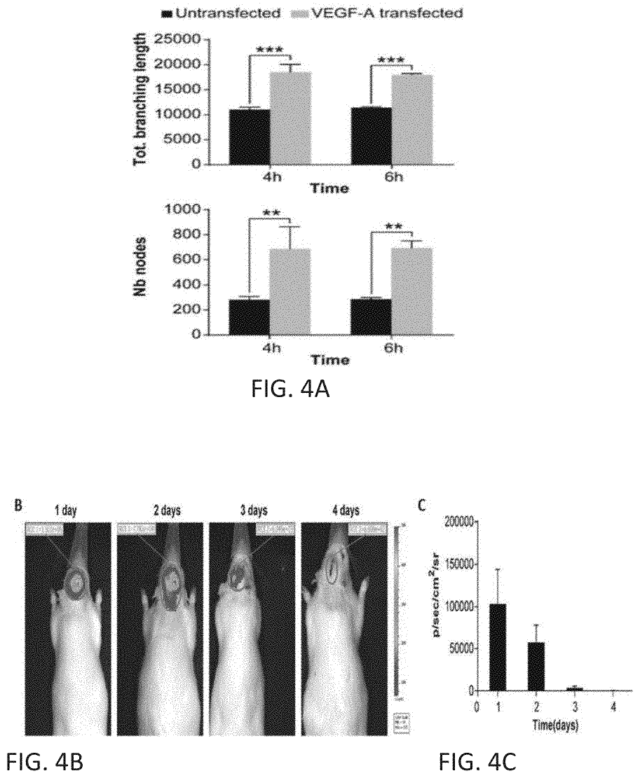

In addition, the potential for modVEGF-A to induce angiogenesis was assessed using an endothelial tube formation assay, shown in FIG. 4. The total branching length and number of nodes in the modVEGF-A transfected group were significantly higher than that of those found in the untransfected group (P<0.05, N=3). The representative images of the tube formation assay revealed that modVEGF-A treated HUVEC cells increased branch length and the numbers of endothelial tubes compared to those in normal culture medium.

### Example 3—Demonstration that Transplantation of Biomaterials Combined with Modified mRNA-Treated BMSCs Enhanced Bone Repair In Vivo

Towards assessing the therapeutic potential of BMSCs harboring two modRNAs—one encoding human BMP-2 and the other encoding human VEGF-A—BMSCs isolated from rats were co-transfected with said modRNAs and prepared with a collagen fiber matrix. The prepared composition was surgically implanted at the orthotropic site in rats following cranial defect surgery. The therapeutic potential of the composition was assessed by imaging the bone and staining the bone to quantify new bone growth and bone repair.

### Methods

Fabrication and Functionalization of Collagen Sponges

Collagen sponges were dissolved in 0.1 mol/L acetic acid and frozen at −80° C., then freeze-dried at −50° C. under a vacuum for 24 hours, subsequently cross-linked by 25 mM EDC and 25 mM NHS with 95% ethanol solution for 24 hours, thoroughly washed with 5 wt % glycine solution and distilled water three times, and freeze-dried a second time at −50° C. to obtain collagen scaffold.

Preparation of Rat modRNA-BMSC/Scaffold Constructs and Surgical Procedures

The modRNA-transfected cells were trypsinized 4 hours post-transfection, centrifuged, and resuspended in osteoblast inducing conditional medium. The cells (5×10⁶ cells/ml) were evenly seeded into cross-linked collagen scaffold in the 48-well plate, 50 μl/scaffold, thus 2.5×10⁵ cells/scaffold. Osteogenic medium was added after 4 hours of incubation at 37° C., and the BMSC/scaffold constructs were incubated overnight prior to implantation (Lo et al., Biomaterials, 124, 1-11 (2017)).

Eight week-old male Sprague Dawley rats were purchased from the animal experimental center of Shanghai ninth people's hospital (China), where they were housed and looked after in the experimental animal house. Two weeks later, the rats were anesthetized by intraperitoneal injection of 10% chloral hydrate (4 mg/kg body weight). In order to expose the parietal bone adequately, a sagittal incision (1.8 cm) in the scalp was made and the pericranium was removed by gentle scraping. The skull was washed by sterile phosphate buffered solution (PBS). Two critical-sized (5 mm in diameter) defects were created on both sides of the sagittal suture on the parietal bone, without disturbing the underlying dura and superior sagittal sinus vein, using a sterile medical bone drill. The constructs were implanted into the defects and gently pressed. The incision was carefully closed in layers by a 5-0 biodegradable suture and a 6-0 silk thread. Five treatment regimens were employed in this study, where defects were implanted with one of the following: 1) empty collagen scaffold (n=6); 2) naïve BMSC loaded collagen scaffold (n=6); 3) BMSCs treated with modRNA encoding VEGF-A -loaded collagen scaffold (n=6); 4) BMSCs treated with modRNA encoding hBMP-2-loaded collagen scaffold (n=6); and 5) BMSCs treated with modRNA encoding hBMP-2+VEGF-A -loaded collagen scaffold (n=6). No antibiotics were given postoperatively but carefully housed in the experimental animal house.

For the luciferase study, collagen scaffolds loaded with BMSCs transfected with modRNA encoding luciferase (n=3) were implanted in the defect site. D-Luciferin, Sodium Salt (CAS 103404-75-7; Yeasen, Shanghai, China) was injected intraperitoneally using a 27 gauge needle at a dosage of 150 mg/kg body-weight under general anesthesia. In the following days, bioluminescence imaging was performed using a Xenogen IVIS Lumina XRMS Series III live animal biophotonic imaging system (Caliper Life Sciences, USA). IVIS images were recorded at 1, 2, 3, and 4 days following surgery in 3 animals per time point. Image analysis was performed using the software Living Image (Caliper, USA).

Micro Computed Tomography (pCT)

Rats were sacrificed and the calvarial bones were removed at 4 and 12 weeks post-surgery, and four samples per group were harvested. The samples were fixed in 4% paraformaldehyde for 24 h, and then were scanned using a SkyScan-1176 micro-computed tomography (μCT) (Bruker micro CT, Belgium) system. Scans were performed using 17.93 voxel size, 90 KV, 278 pA and 0.5 degrees rotation step (180 degrees angular range). For bone analysis, micro-CT evaluation was performed on a 5 mm diameter circle, 1 mm height cylinder region in the defect area. The 1.6 version of NR econ software was used for 3D reconstruction and viewing of images. After 3D reconstruction, the 1.13 version of CT software was used for bone analysis. The index including directly measured bone volume (BV, mm³), tissue volume (TV, mm³), and the ratio BV/TV (%), were calculated for the bone formed in the defect area.

Histological and Immunohistochemical Staining

After μCT scanning, the removed calvarial bones were decalcified with mild EDTA-decalcifier-solution (Boster Biological Technology Co., Ltd.), followed by paraffin embedding and sectioning. The sections (10 mm thick) from mid-defect regions were stained with H&E or subjected to Masson trichrome staining, which detects the collagen formation activity and is a sign of bone remodeling, that is, an essential process to complete bone healing. Briefly, the sections were deparaffined and rehydrated for immunohistochemical staining of osteocalcin (OCN) or CD31. The antigen retrieval was performed by incubation with trypsin for 20 minutes at 37° C. The primary antibodies used were mouse anti-rat OCN (1:150 dilution, abcam) or anti-CD31 MAb (1:200 dilution, Servicebio). The secondary antibody used was goat anti-mouse HRP-conjugated MAb (1:1000 dilution, Abcam). For Masson, OCN, and CD31 staining, the nuclei were stained with Gill Hematoxylin (Sigma) at the end of procedures according to the manufacturer's instructions.

Gene Expression Analysis by qRT-PCR

Newly formed bone tissues in the defect areas of the crania were sampled after the rats were euthanized 4 and 12 weeks after surgery. Total RNA was extracted from the bone tissue using a trizol reagent (Invitrogen, CA) according to the manufacturer's instructions. Then, the uniform real-time (RT) primer was used for the reverse transcription and quantitative RT-PCR was performed using a real-time thermal cycler (Stratagene Mx3000P™ QPCR System, CA, USA) and 2×SYBR Green PCR master mix (EZBioscience, MN, USA).

Western Blot Analysis

Whole bone tissue isolations were extracted using a total protein lysis buffer. Tissue samples were lysed in RIPA lysis buffer (Sigma-Aldrich) containing a proteasome inhibitor (Beyotime) and then followed by western blot as previously described (El Rashidy et al., Acta Biomater., 2017 Oct. 15; 62:1-28). Finally, the target bands were acquired with an ECL Plus Western Blotting Detection System (GE Healthcare, IL, USA).

### Results

To evaluate the in vivo protein expression kinetics following the introduction of modRNA-treated BMSCs, bioluminescence imaging was performed. At 24 hr time intervals following implantation, animals were sedated and non-invasively imaged. Luciferase expression was detected and peaked after 1 day with an average value of 1.0×10⁵ photons/sec/cm²/sr. Signal intensities declined gradually over the course of 3 days (FIG. 4), but maintained a signal over baseline (remaining activity of 6.6×10² photons/sec/cm²/sr on day 3 post-treatment). However, after 4 days luciferase activity dropped to baseline values.

To assess for efficacy, cranial defect surgery was performed on rats as previously described (El-Rashidy et al., Acta Biomater., 62:1-28 (2017)). A collagen fiber matrix was loaded with modRNA-transfected BMSCs and surgically implanted at the orthotropic site (FIGS. 5A, 5B). The rats were sacrificed at 4 and 12 weeks post-operation, where levels of bone healing were evaluated through histology or by using micro-computed tomography (μCT) scans to calculate bone volume (BV) and percent bone volume (BV/TV).

The combinatorial treatment of BMP2+VEGF-A modRNA was most capable of generating new bone tissue deposition that rapidly filled the defect space at both 4 and 12 weeks compared to the individual treatment of BMP2 modRNA or VEG-A modRNA (FIG. 5C) at half the dosage/concentration used for the BMP-2 and VEGF-A delivered as individual polypeptides. Quantitative analysis of μCT scans was applied to evaluate the regenerated bone volume (BV) and the ratio of BV/TV. When quantified, the BMP2+VEGF-A modRNA treatment group increased neo-bone formation over all other groups both at 4 and 12 weeks (P<0.01), and gave rise to higher BV/TV ratios than single treatments or collagen treatment only (FIGS. 5D-5G). The BV/TV was 2.7-fold higher in defects treated with the BMP2+VEGF-A modRNA-loaded collagen scaffold, when compared to the untreated group, 2.45-fold higher than the modVEGF-A treated group, and 1.59-fold higher than the modBMP2 treated group at 12 weeks, respectively.

Thus, the combination therapy as described herein provided a synergistic effect compared to the single polypeptide dosing, because only half the delivered dose of the BMP-2 and VEGF-A in the combination was clearly more effective at regenerating and healing the bone defect than either polypeptide delivered to the bone defect alone.

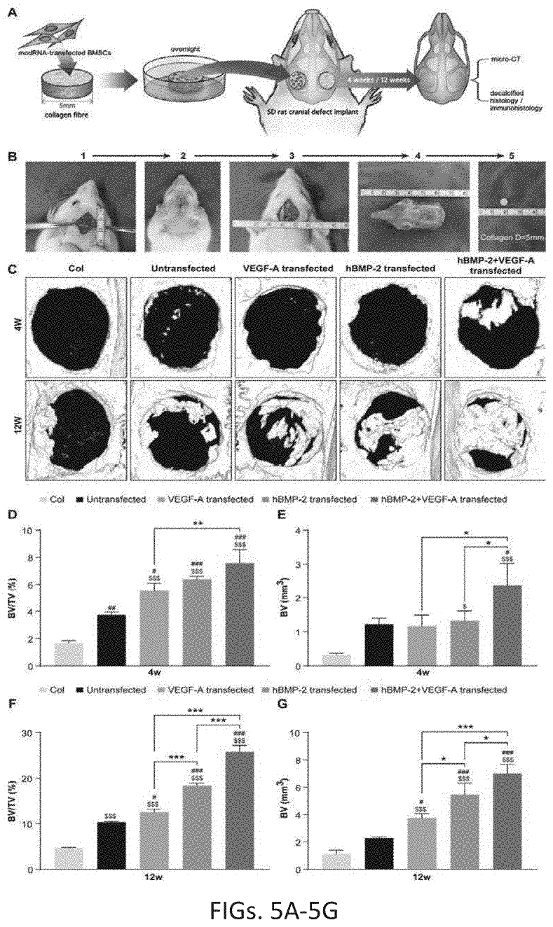

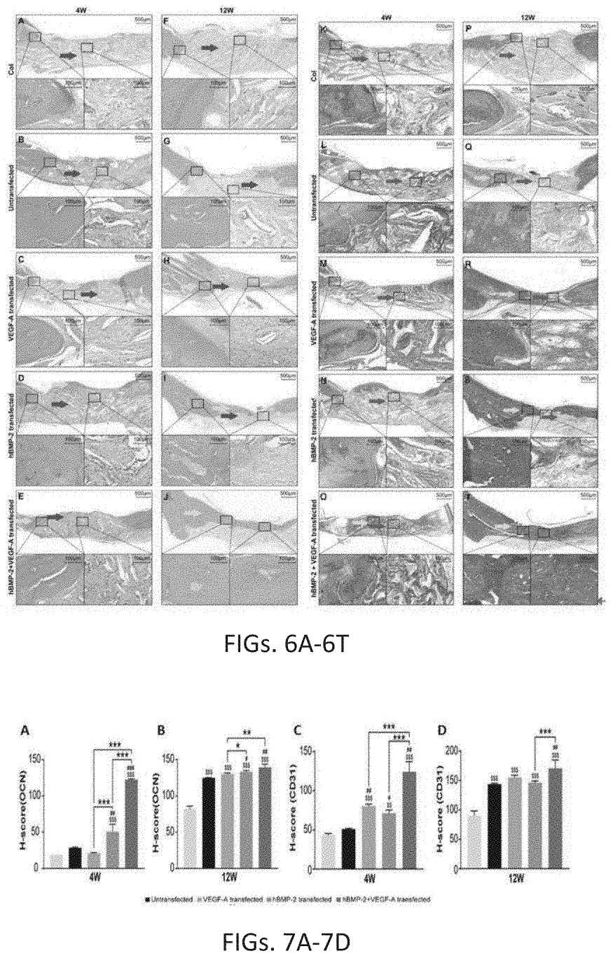

Histological staining was performed to evaluate bone regeneration from the modRNA-transfected collagen scaffolds at week 4 and 12 post-surgery and transplantation. Distinct patterns of orthotopic bone tissue formation were observed following the administration of different modRNA-transfected BMSC scaffolds. The BMP2+VEGF-A modRNA transfection group acquired the largest area of newly regenerated bone tissue, compared to all other treatment and control groups (FIGS. 4A-4G). Hematoxylin and eosin staining revealed a dark red homogeneous and organized bone tissue formation in the bone defect of the BMP2+VEGF-A modRNA transfected group (FIGS. 6E, 6J).

In contrast, histological analysis from the control groups, those either treated with empty collagen or receiving no treatment, presented with negligible neobone formation and instead stained pink from fibrous connective tissue (FIGS. 6 A, F, B, G). In single treatment groups, part of the bone defect area was occupied by dark red homogeneous stained bone tissue, and part of the bone defect area was occupied by pink dyed fibrous connective tissue indicating limited and incomplete repair (FIGS. 6 C, H, D, I). Similarly, Masson's staining of the BMP2+VEGF-A modRNA treatment group displayed large amounts of dark blue homogeneous bone tissue, indicating an advanced phase of bone tissue formation in the defect area (FIGS. 6 O, T). In the collagen only group and the untransfected scaffold groups the defect was covered with light blue fibrous connective tissue (FIGS. 6 K, P; L, Q, respectively); and the single treatment groups again gave rise to limited and partial bone repair (FIGS. 6 M, R; N, S).

To further characterize how the modRNA-transfected BMSCs stimulated neobone formation, immunohistochemical (IHC) analysis was performed. Briefly, tissue sections were stained for osteocalcin (OCN) at 4 and 12 weeks post-surgery and treatment. Both the non-transfected control group as well as the acellular treatment group failed to provide positive staining for OCN at 4 weeks, with remote negligible expression of OCN at 12 weeks post-surgery. OCN was found to be up-regulated in the modBMP2 and BMP2+VEGF-A modRNA groups as early as 4 weeks post treatment, indicating efficient and effective osteogenic stimulation from these two treatment regimens. At 12 weeks post treatment, positive staining for OCN was observed in all the groups, however OCN activity increased significantly in the BMP2+VEGF-A modRNA group suggesting that this combination has higher osteogenic compatibility. Quantitative analysis further supported these findings (FIGS. 7A, 7B).

Histological evaluation of CD31 revealed weak and inconsistent signals in the control and acellular treatment groups, indicating poor neovascularization following these treatments. The treatment groups exhibited positive staining of CD31 at 4 weeks and 12 weeks post-transplantation, with varying intensities. When quantified, significantly higher abundances of CD31⁺ blood vessels were found in the BMP2+VEGF-A modRNA group (FIGS. 7C, 7D). These findings suggest that the combined treatment of BMP-2 and VEGF-A synergistically improve calvarial bone formation potentially through neovascularization mechanisms.

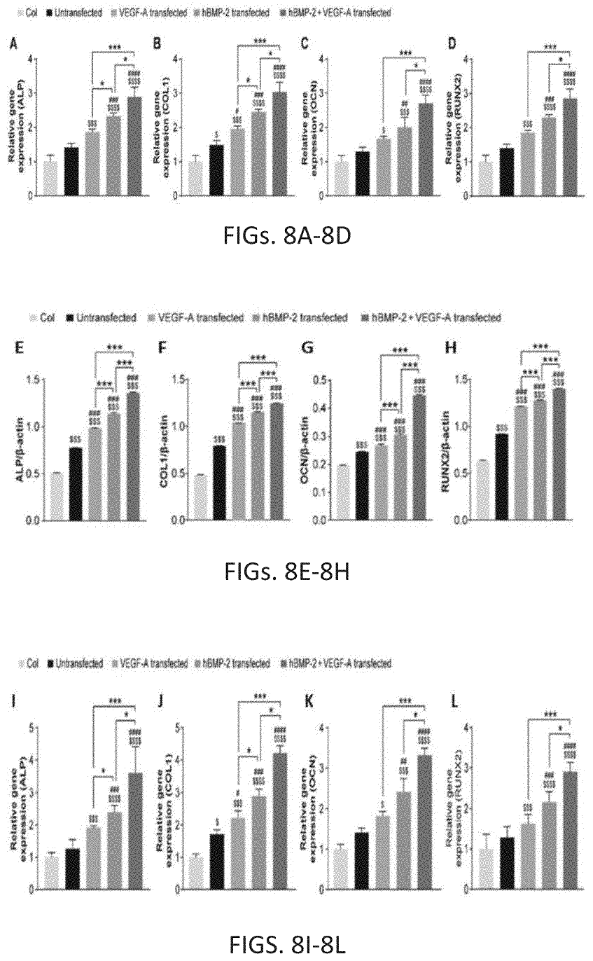

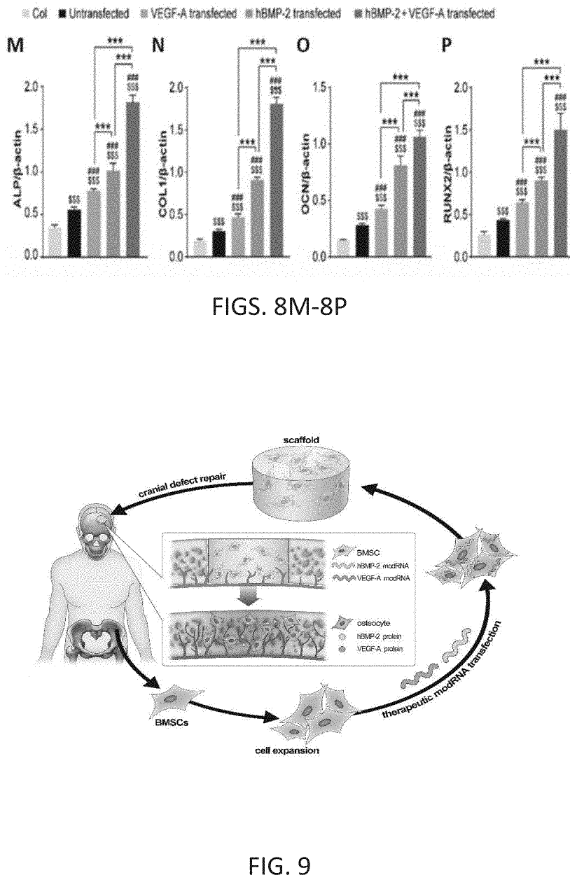

To investigate the potential of our modRNA-BMSC materials to act on genes that regulate or promote bone formation, RNA and protein was analyzed from regenerating bone tissue from all treatment groups (FIG. 8). All the modRNA treatment groups consistently stimulated heightened gene expression patterns of osteogenic genes at 4 wk and 12 wks post-surgery (FIGS. 8A-8D, FIGS. 8I-8L), compared to the acellular group and non-transfection group (p<0.05, n=3). Similarly, western blot analysis showed that these osteogenic genes were also up-regulated on the protein level, when compared to control groups (FIGS. 8E-8H, 8M-8P). Among the three transfected groups, the BMP2+VEGF-A modRNA treatment group induced the highest gene expression levels and most significantly elevated protein levels of the osteogenic genes. Consequently, BMP2 and VEGF-A modRNA stimulate endogenous bone repair/growth mechanisms thus accelerating bone tissue regeneration synergistically (FIG. 9).

### Example 4—Demonstration that BMP and VEGF-A Modified mRNA-Treated BMSCs Induce Favorable Osteo-Inductive Properties at Different Ratios, Over Recombinant Proteins In Vitro and In Vivo

To assess the osteo-inductive effects of BMSCs harboring two modRNAs at different ratios (one encoding human BMP-2 and the other encoding human VEGF-A), BMSCs isolated from rats were co-transfected with these modRNAs or treated with recombinant human proteins (rh proteins) of these genes. The transfected BMSCs were cultured in the absence of dexamethasone (a known osteogenic compound) where mineralization and calcium deposition was assessed together with gene expression profiles of known osteogenic markers. Later a composition including modRNA or rh protein-treated BMSCs together with a collagen fiber matrix were surgically implanted at the orthotopic site in rats following cranial defect surgery. The therapeutic potential of the composition was assessed by imaging the bone and staining the bone to quantify new bone growth and bone repair.

### Methods

In vitro Assessment of Osteogenesis in the Absence of Dexamethasone

Alkaline phosphatase activity assay and staining was carried out after osteogenic induction culture for 7 days. Alizarin red staining and quantification was carried out to detect mineralization at 14 days post transfection. Molecular analysis for major osteogenic genes were carried out using qRT-PCR and western blotting at 7 days post transfection. BMSCs were treated with different ratios of modified mRNAs, namely 1:1 ratio of BMP-2 to VEGF-A (BV), a 3:1 ratio of BMP-2 to VEGF-A (3B1V), a 1:3 ratio of BMP-2 to VEGF-A (1B3V), or treated with recombinant human proteins, i.e., recombinant human protein BMP (rB), recombinant human protein VEGF (rV) or both (rBV), and compared against control groups which were either BMSCs transfected with an inert modRNA construct, Luciferase (Luc), or naive untreated BMSCs (ctrl).

Fabrication and Functionalization of Collagen Sponges

Collagen sponges were partially dissociated in 0.1 mol/L acetic acid and frozen at -80° C., then freeze-dried at −50° C. under a vacuum for 24 hours, subsequently cross-linked by 25 mM EDC and 25 mM NHS with 95% ethanol solution for 24 hours, thoroughly washed with 5 wt % glycine solution and distilled water three times, and freeze-dried a second time at −50° C. to obtain a collagen scaffold.

Preparation of Rat modRNA-BMSC/Scaffold Constructs and Surgical Procedures

The modRNA-transfected cells were trypsinized 4 hours post-transfection, centrifuged, and resuspended in osteoblast inducing conditional medium. The cells (5×10⁶ cells/ml) were evenly seeded into cross-linked collagen scaffold in the 48-well plate, 50 μl/scaffold, thus 2.5×10⁵ cells/scaffold. Osteogenic medium was added after 4 hours of incubation at 37° C., and the BMSC/scaffold constructs were incubated overnight prior to implantation (Lo et al., Biomaterials, 124, 1-11 (2017)).

Eight week-old male Sprague Dawley rats were housed in an experimental animal house. Two weeks later, the rats were anesthetized by intraperitoneal injection of 10% chloral hydrate (4 mg/kg body weight). To expose the parietal bone adequately, a sagittal incision (1.8 cm) in the scalp was made and the pericranium was removed by gentle scraping. The skull was washed by sterile phosphate buffered solution (PBS). Two critical-sized (5 mm in diameter) defects were created on both sides of the sagittal suture on the parietal bone, without disturbing the underlying dura and superior sagittal sinus vein, using a sterile medical bone drill. The constructs were implanted into the defects and gently pressed. The incision was carefully closed in layers by a 5-0 biodegradable suture and a 6-0 silk thread.

Four treatment regimens were employed in this study, where defective areas received implants with one of the following: 1) BMSCs treated with modRNA encoding Luciferase-loaded into a collagen scaffold 2) BMSCs treated with modRNA encoding hBMP2:VEGFA(in a 3:1 ratio)-loaded into a collagen scaffold 3) BMSCs treated with modRNA encoding hBMP-2:VEGF-A (in a 1:3 ratio)-loaded into a collagen scaffold and 4) BMSCs treated with recombinant protein BMP-2+VEGFA (1:1) loaded into a collagen scaffold.

Micro Computed Tomography (μCT)

Rats were sacrificed and the calvarial bones were removed at 4 weeks post-surgery, and n=3 samples per group were harvested. The samples were fixed in 4% paraformaldehyde for 24 h, and then were scanned using a SkyScan-1176 micro-computed tomography (μCT) (Bruker micro CT, Belgium) system. Scans were performed using 17.93 μm voxel size, 90 KV, 278 μA and 0.5 degrees rotation step (180 degrees angular range). For bone analysis, micro-CT evaluation was performed on a 5 mm diameter circle, 1 mm height cylinder region in the defect area. The 1.6 version of NR econ software was used for 3D reconstruction and viewing of images. After 3D reconstruction, the 1.13 version of CT software was used for bone analysis. The index including directly measured bone volume (BV, mm³), tissue volume (TV, mm³), and the ratio BV/TV (%), were calculated for the bone formed in the defect area.

Histological and Immunohistochemical Staining

After μCT scanning, the removed calvarial bones were decalcified with mild EDTA-decalcifier-solution (Boster Biological Technology Co., Ltd.), followed by paraffin embedding and sectioning. The sections (10 mm thick) from mid-defect regions were stained with H&E or subjected to Masson trichrome staining, which detects the collagen formation activity and is a sign of bone remodeling, that is, an essential process to complete bone healing. Briefly, the sections were deparaffined and rehydrated for immunohistochemical staining of osteocalcin (OCN) or CD31. The antigen retrieval was performed by incubation with trypsin for 20 minutes at 37° C. The primary antibodies used were mouse anti-rat OCN (1:150 dilution, abcam) or anti-CD31 MAb (1:200 dilution, Servicebio). The secondary antibody used was goat anti-mouse HRP-conjugated MAb (1:1000 dilution, Abcam). For Masson, OCN, and CD31 staining, the nuclei were stained with Gill Hematoxylin (Sigma) at the end of procedures according to the manufacturer's instructions.

Gene Expression Analysis by qRT-PCR

Newly formed bone tissues in the defect areas of the crania were sampled after the rats were euthanized 4 and 12 weeks after surgery. Total RNA was extracted from the bone tissue using a trizol reagent (Invitrogen, CA) according to the manufacturer's instructions. Then, the uniform real-time (RT) primer was used for the reverse transcription and quantitative RT-PCR was performed using a real-time thermal cycler (Stratagene Mx3000P™ QPCR System, CA, USA) and 2×SYBR Green PCR master mix (EZBioscience, MN, USA).

Western Blot Analysis

Whole bone tissue isolations were extracted using a total protein lysis buffer. Tissue samples were lysed in RIPA lysis buffer (Sigma-Aldrich) containing a proteasome inhibitor (Beyotime) and then followed by western blot as previously described (El Rashidy et al., Acta Biomater., 2017 Oct. 15; 62:1-28). Finally, the target bands were acquired with an ECL Plus Western Blotting Detection System (GE Healthcare, IL, USA).

### Results

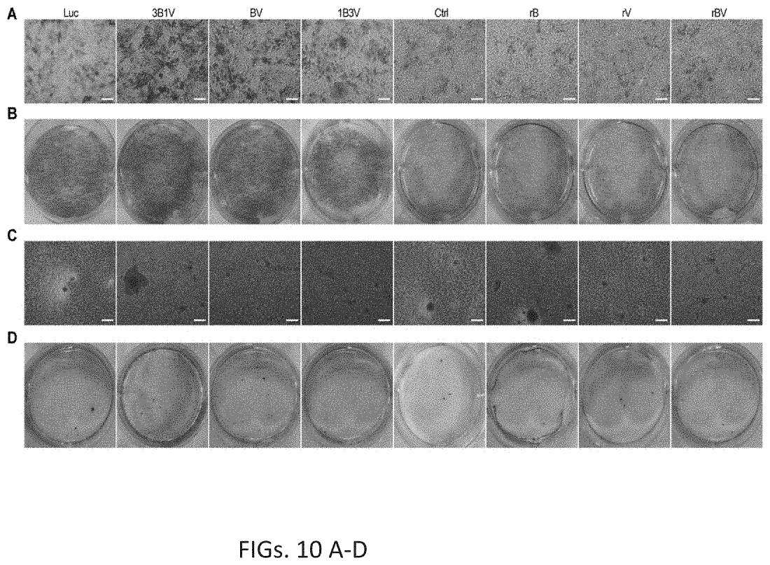

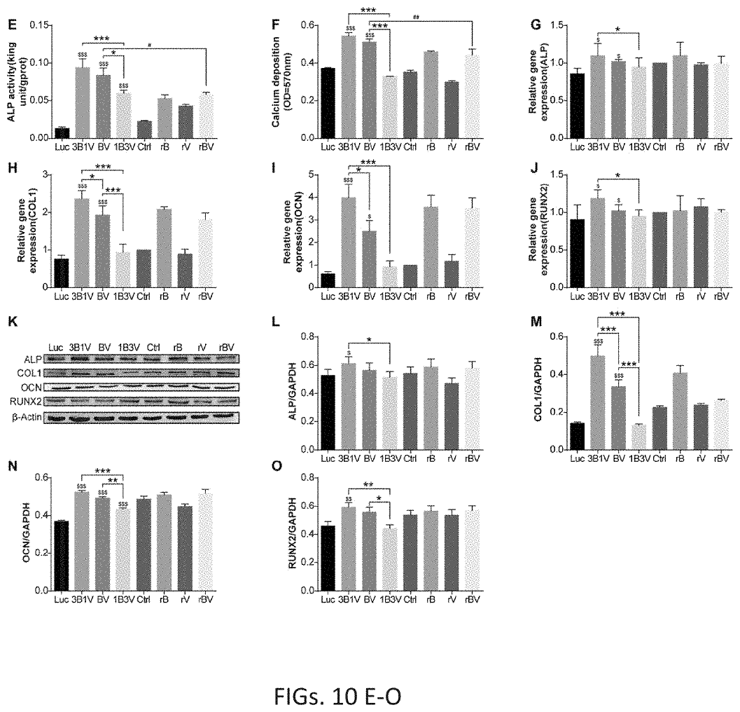

Evaluation of osteo-inductive activity in the absence of dexamethasone was analyzed in BMSCs undergoing different modRNA or recombinant protein treatments. Microscopic evaluation showed increased ALP and calcium deposition stemming from the 3B1V (3:1 BMP to VEGF ratio) treatment group as well as the 1:1 (BV) treatment ratio as compared to control groups (Ctrl, Luc) and recombinant protein groups (single or double treatment groups i.e. rB, rV and rBV) (see FIGS. 10A-D, which show the power of the mRNAs and protein, specifically the ability to drive MSCs into an osteogenic lineage in the absence of dexamethasone (a known osteogenic chemical used widely in bone differentiation protocols)). A quantified significant statistical improvement in heightened levels of ALP activity and calcium deposition was observed in both the BV as well as the 3B1V treatment groups over the controls and recombinant protein groups (FIGS. 10 E-F). In addition, the modRNA treatment groups, namely BV and 3B1V performed equally as well or better than the recombinant protein groups when investigating osteogenic gene expression and stimulation (FIGS. 10 G-O). mRNA levels of ALP, COL1, OCN and RUNX2 were all statistically increased when employing ratios of BMP to VEGF at a 3B1V or BV ratio compared to control groups and 1B3V groups (FIGS. 10G-J). Similarly, on the protein level, these genes saw significant increases when treated with the 3B1V and BV ratios over 1B3V ratios and control groups (FIGS. 10 K-O).

A collagen fiber matrix was loaded with modRNA-transfected or protein treated BMSCs and surgically implanted at the orthotropic site (as previously shown in FIG. 5). The rats were sacrificed at 4 weeks post-operation, where levels of bone healing were evaluated through histology or by using micro-computed tomography (μCT) scans to calculate percent bone volume (BV/TV).

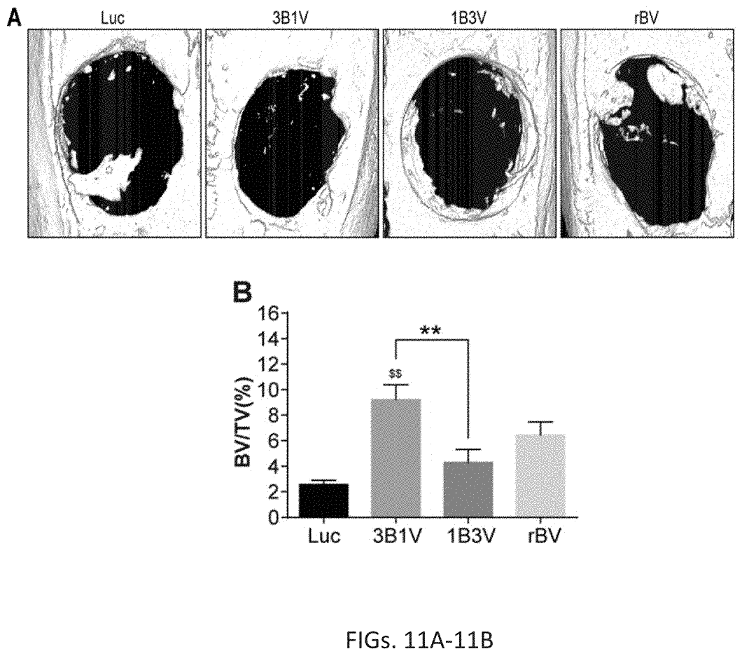

The combinatorial treatment of BMP2+VEGF-A modRNA at a ratio of 3:1 (3B1V) was most capable of generating new bone tissue deposition that partially filled the defect space as early as 4 weeks post-implantation, compared to the recombinant protein treatment groups, controls, and alternative ratios (i.e., 1B3V), (FIG. 11A, which shows bone ingrowth, where the smallest dark area is the best result). Quantitative analysis of μCT scans was applied to evaluate the regenerated bone volume (BV) as a ratio of BV/TV. When quantified, the 3B1V modRNA treatment group increased neo-bone formation over all other groups at 4 weeks (P<0.01), and gave rise to higher BV/TV ratios than recombinant protein groups, alternative ratios, or control groups (FIG. 11B). The BV/TV was 3.5-fold higher in defects treated with the 3B1V modRNA-loaded collagen scaffold, when compared to the untreated group.

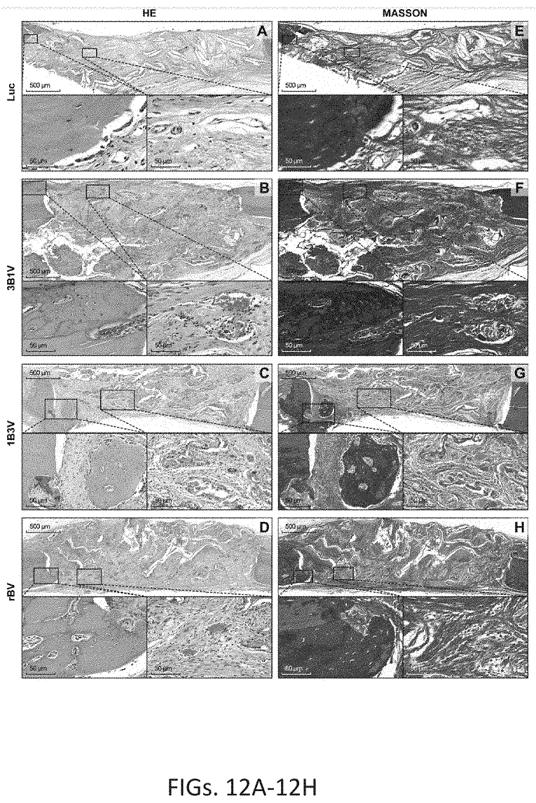

Histological staining in the form of H&E and Masson's staining were performed to evaluate bone regeneration from the modRNA-treated or recombinant protein-treated BMSCs in collagen scaffolds at 4 weeks post-surgery and transplantation. The histological evaluation revealed that the 3B1V treatment group gave rise to distinct patterns of orthotopic bone tissue formation. Specifically, the 3B1V treatment gave rise to more bony union structure and the appearance of significantly more osteoblast-like cells in the underlying matrix (FIGS. 12A-D).

In contrast, histological analysis from the control (Luciferase treated) group, and 1B3V ratio group presented with negligible neobone formation and instead stained pink from fibrous connective tissue (FIGS. 12A-C). In the recombinant protein treatment group, part of the bone defect area was occupied by dark red homogeneous stained bone tissue, and part of the bone defect area was occupied by pink dyed fibrous connective tissue indicating limited and incomplete repair (FIG. 12D).

Further, the 3B1V treatment group gave rise to more densely contained collagen fibers, an indication of more complete bone growth, as made evident by regions of dark blue Masson's staining (FIGS. 12 E-H). In the Luciferase-treated group and 1B3V ratio treatment group the defects were covered with light blue fibrous connective tissue (FIGS. 12E-12G), respectively, and the recombinant protein treatment group again gave rise to less advanced bone regeneration (FIG. 12H).

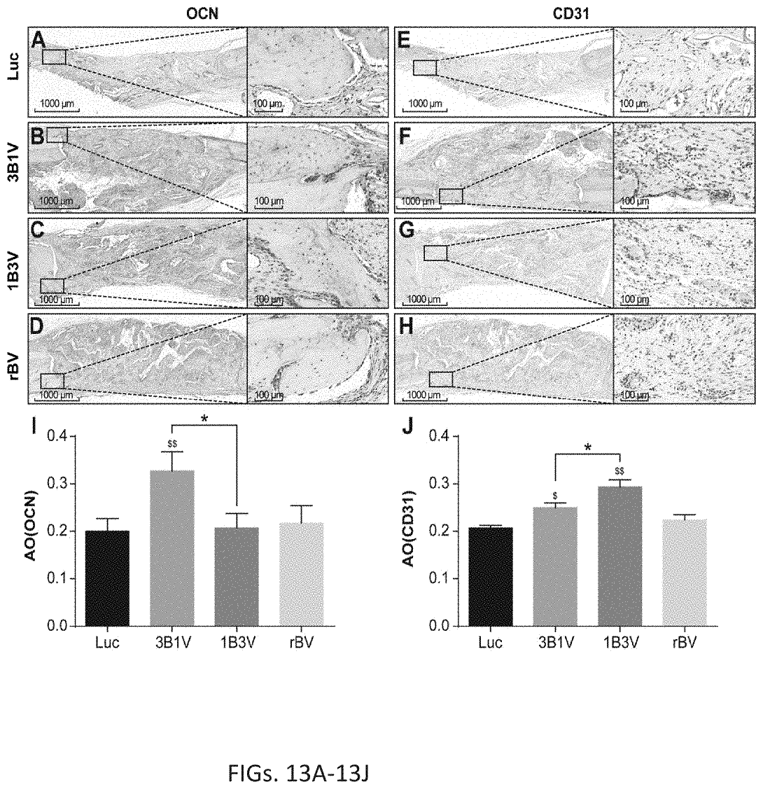

To further characterize how the different ratios of BMP and VEGF modRNA-transfected BMSCs stimulated neobone formation, immunohistochemical (IHC) analysis was performed. Briefly, tissue sections were stained for osteocalcin (OCN) at 4 weeks post-surgery and treatment. Increased levels of OCN were verified in the 3B1V treatment group over controls and significantly higher than the 1B3V ratio group (FIGS. 13A-D, FIG. 13I). The recombinant protein treatment group provided satisfactory expression of OCN at 4 weeks post-surgery. Quantitative analysis further supported these findings (FIG. 13 I). Furthermore, histological evaluation of CD31 revealed weak and inconsistent signals in both the Luciferase modRNA control group as well as the recombinant protein group, indicating poor neovascularization following these treatments (FIGS. 13E-H).

Both of the 3B1V and 1B3V treatment groups exhibited positive staining of CD31 at 4 weeks post-transplantation, with varying intensities (FIGS. 13 F-G). When quantified, significantly higher abundances of CD31⁺ blood vessels were found in the 1B3V group (FIG. 13J). These findings suggest that altering ratios of BMP-2 and VEGF-A modRNAs synergistically improve calvarial bone formation potentially through neovascularization mechanisms and may more favorably drive neobone formation.

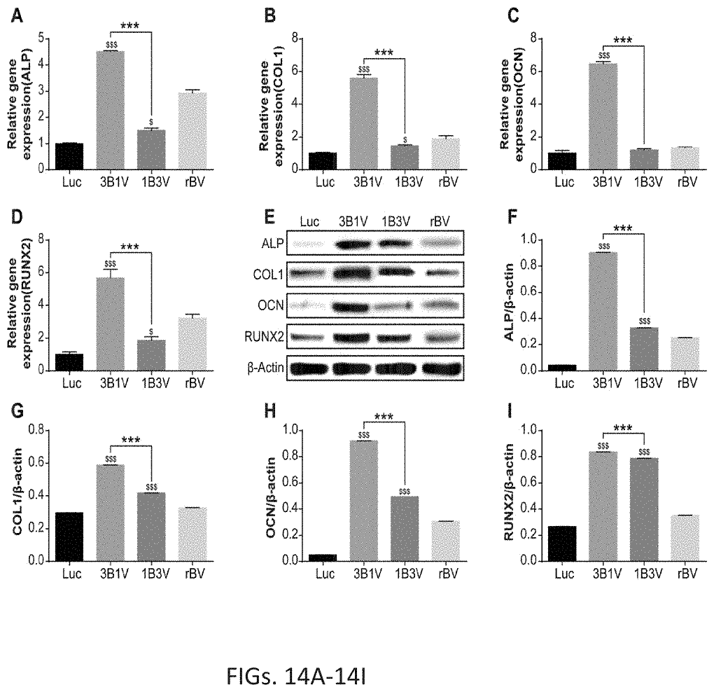

To investigate the heightened potential of our modRNA-BMSC materials to act on genes that regulate or promote bone formation, RNA and protein were analyzed from regenerating bone tissue from these different modRNA ratio treatments and compared to those levels found from recombinant protein or control treatment groups (FIGS. 14A-I). When assessing gene expression profiles at the mRNA level of ALP, COL1, OCN, and RUNX2 at 4 weeks post transplantation, the 3B1V treatment group gave statistically significant increases in expression of these marker genes within the newly forming bone tissue compared to the luciferase control group, 1B3V and recombinant protein group (FIGS. 14A-D).

Similarly, when assessing gene expression at the protein level of these osteogenic marker genes, again significant increases in gene expression were noted from the 3B1V treatment group compared with the 1B3V group as well as the modLuc and recombinant protein control groups (FIGS. 14E-I). Consequently, a ratio of 3B1V modRNA seems to best stimulate endogenous bone repair/growth mechanisms thus accelerating bone tissue regeneration synergistically.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising
(a) a mesenchymal stem or stromal cell (MSC) composition comprising either one or both of:
(i) a plurality of MSCs, wherein each MSC harbors one or more modified RNA molecules encoding bone morphogenetic protein (BMP) and one or more modified RNA molecules encoding vascular endothelial growth factor (VEGF), or
(ii) first and second separate pluralities of MSCs, wherein each MSC in the first plurality of MSCs harbors one or more modified RNA molecules encoding bone morphogenetic protein (BMP), and wherein each MSC in the second plurality of MSCs harbors one or more modified RNA molecules encoding vascular endothelial growth factor A (VEGF-A); and
(b) a carrier.

2. The composition of claim 1, wherein the carrier comprises a gel, hydrogel, paste, or a solid carrier.

3. The composition of claim 2, wherein the carrier comprises one or more of a bioceramic, bioactive glass, polymer, nanofiber, nanomaterial, hydroxyapatite, collagen, non-toxic lipid nanoparticles, or a gel-based or silica-based hydrogel.

4. The composition of claim 1, wherein the VEGF polypeptide is a human VEGF-A polypeptide and the BMP polypeptide is a human BMP-2 polypeptide.

5. The composition of claim 1, wherein the MSCs are bone marrow-derived MSCs (BMSCs), adipose/fat-derived MSCs, peripheral blood-derived MSCs, umbilical cord-derived MSCs, or placental-derived MSCs.

6. The composition of claim 1, wherein the MSCs are allogenic cells or autologous cells.

7. The composition of claim 1, wherein the MSCs harboring the modified RNA molecules are prepared by (i) transient transfection or electroporation with the modified RNA molecules or (ii) exposure to lipid nanoparticles comprising the modified RNA molecules.

8. The composition of claim 1, wherein the total amount of the modRNAs used in the composition is in the range of 100 ng to 12 mg.

9. The composition of claim 1, wherein the total number of cells in the composition is in the range of about 20×10⁴ to about 120×10⁶.

10. The composition of claim 1, wherein the dose of mRNA transcripts within the MSCs is about 1 pg/cell to 1 ng/cell.

11. The composition of claim 1, wherein the composition comprises about 25 μg to about 2.5 mg of modRNA in about 250,000 cells to about 25×10⁶ cells at a dose of about 10 pg/cell, and secretes about 10 ng to about 1 mg of the BMP and VEGF polypeptides over a period of 5 days.

12. The composition of claim 1, wherein a ratio of BMP to VEGF is in a range of 10:1 to 1:1.

13. The composition of claim 1, wherein the ratio of BMP-VEGF is about 3:1 to about 6:1.

14. A method of treating a bone defect in a subject, the method comprising
identifying a subject with a bone defect; and
administering to the bone defect a composition of claim 1.

15. The method of claim 14, wherein the bone defect comprises a site of osteoporosis, a bone tumor, a bone break, a site of bone trauma, a nonunion bone fracture, a bone tumor resection, or a bone affected by craniomaxillofacial surgery.

16. The method of claim 14, wherein the carrier comprises a gel, hydrogel, paste, or a solid carrier.

17. The method of claim 14, wherein the VEGF polypeptide is a human VEGF-A polypeptide and the BMP polypeptide is a human BMP-2 polypeptide.

18. The method of claim 14, wherein the MSCs are bone marrow-derived MSCs (BMSCs), adipose/fat-derived MSCs, peripheral blood-derived MSCs, umbilical cord-derived MSCs, or placental-derived MSCs.

19. The method of claim 14, wherein the MSCs are allogenic cells or autologous cells.

20. The method of claim 14, wherein the composition is administered by one or more of the following:
(i) injection to the bone defect;
(ii) vascular catheterization;
(iii) using MSCs engineered to migrate to a site of the bone defect; or
(iv) a surgical procedure to render access to the bone defect.
